(19) United States
(12) Patent Application Publication
(10) Pub. No.: US 2011/0124683 A1
(43) Pub. Date: May 26, 2011

(54) Use of CRTH2 Antagonist Compounds

(75) Inventors: Michael George Hunter, Abingdon (GB); Eric Roy Pettipher, Abingdon (GB); Colin Michael Perkins, Abingdon (GB); Mark Anthony Payton, Abingdon (GB); Luzheng Xue, Abingdon (GB)

(73) Assignee: Oxagen Limited, Abingdon (GB)

(21) Appl. No.: 12/779,638

(22) Filed: May 13, 2010

Related U.S. Application Data
(63) Continuation-in-part of application No. PCT/GB2008/003824, filed on Nov. 13, 2008.
Application No. PCT/GB2008/003824 is a continuation-in-part of application No. PCT/GB2008/003843, filed on Nov. 13, 2008.

(30) Foreign Application Priority Data
Nov. 13, 2007 (GB) 0722203.7
Nov. 13, 2007 (GB) 0722216.9

(51) Int. Cl. A61K 31/405; A61K 31/4709; A61K 31/4439; A61P 37/08

(52) U.S. Cl. 514/314; 514/419; 514/339

(57) ABSTRACT

The invention relates to compounds of general formula (I):

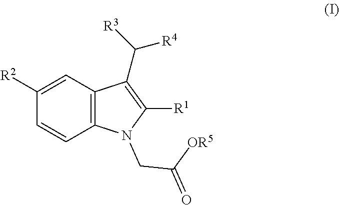

wherein R¹, R², R³, R⁴ and R⁵ are as defined herein
for the treatment of allergic conditions, wherein the treatment is by pulsed therapy which comprises a first period during which the compound is administered to the patient and a second period of at least seven days during which the compound is administered to the patient in a reduced amount. The invention also relates to compounds of general formula (I) for desensitizing the immune system of a subject to allergens, thus preventing or reducing the symptoms of allergic conditions such as allergic asthma, allergic rhinitis, or atopic dermatitis.

### BACKGROUND OF THE INVENTION

The present application is a continuation-in-part of PCT/GB2008/003824, filed Nov. 13, 2008. International Application No. PCT/GB2008/003824 claims priority to Great Britain Application No. 0722203.7, filed Nov. 13, 2007. The present application is also a continuation-in-part of International Application No. PCT/GB2008/003843, filed Nov. 13, 2008. International Application No. PCT/GB2008/003843 claims priority to Great Britain Application No. 0722216.9, filed Nov. 13, 2007. The disclosures of these applications are fully incorporated by reference herein.

### FIELD OF THE INVENTION

The present invention relates to the use of CRTH2 antagonist compounds for the pulsed treatment of allergic conditions. The present invention also relates to the use of CRTH2 antagonist compounds for desensitising a patient to allergens, especially to the use of these compounds for the prophylaxis of allergic conditions.

### RELATED ART

Allergic conditions are becoming more common throughout the developed world with as much as 10% of the world's population being affected by one or more of allergic asthma, allergic rhinitis, atopic dermatitis and other allergic conditions. Numerous classes of compound have been suggested for the treatment of these conditions, with one of the more recent developments being the use of CRTH2 antagonists, which inhibit the action of agonists such as prostaglandin D₂ (PGD₂) acting at the CRTH2 receptor.

In patients who are affected by an allergic condition, the presence of an allergen to which the patient is sensitive induces the production of allergen-specific IgE antibodies by B cells, which reach the mature state in which they produce IgE with assistance from T cells, primarily Th2 cells. The allergen-specific IgE is present in the circulation and is also expressed on the surfaces of mast cells and basophils, which release inflammatory substances such as histamine, prostaglandin D₂ and tryptase when the allergen binds to the IgE.

Thus, allergy can be characterised by an increase in atopy, which involves an increase in the number of cells reactive to allergens present in the circulation and tissues of a patient and which may lead to the presence in the circulation and on the surfaces of mast cells and basophils of IgE specific for an allergen to which a patient is sensitive.

There are various approaches to the treatment and prevention of allergy and these include avoidance of the allergen, treatment of the symptoms and desensitisation of a patient to an allergen.

Of these, the first approach may be attempted, for example in cases such as food allergies, but it often presents unacceptable restrictions on the lifestyle of patients.

Desensitisation is also used in a number of cases and, for example, small doses of an allergen may be administered to a patient in order to desensitise the patient to that particular allergen. The approach has been limited by the fact that it is usually necessary for patients to undergo prolonged courses of treatment with the risk of anaphylactic shock and perhaps more importantly by the fact that there is currently no generalised desensitisation treatment that reduces the atopic state to all allergens. With current desentisation regimes it is necessary to personalise the treatment so that each patient is desensitised to the particular set of allergens to which he or she is sensitive.

One example of a document relating to desensitisation treatment is US2004/0265342 (also published as EP 1044019) in which a peptide derived from a polypeptide allergen is administered to a patient.

WO 2004/047793 relates to a solid allergen dosage faun for use in desensitising a patient to the allergen. This approach overcomes some of the problems of a prolonged treatment regime.

Both of these approaches, however, still suffer from the disadvantage that the treatment must be tailored to each individual patient.

Because of the difficulties with the desensitisation approach, much attention has been given to the treatment of the symptoms of allergic conditions.

PGD₂ is an eicosanoid, a class of chemical mediator synthesised by cells in response to local tissue damage, normal stimuli or hormonal stimuli or via cellular activation pathways. Eicosanoids bind to specific cell surface receptors on a wide variety of tissues throughout the body and mediate various effects in these tissues. PGD₂ is known to be produced by mast cells, macrophages and Th2 lymphocytes and has been detected in high concentrations in the airways of asthmatic patients challenged with antigen (Murray et al, (1986), N. Engl. J. Med. 315: 800-804). Instillation of PGD₂ into airways can provoke many features of the asthmatic response including bronchoconstriction (Hardy et al, (1984) N. Engl. J. Med. 311: 209-213; Sampson et al, (1997) Thorax 52: 513-518) and eosinophil accumulation (Emery et al, (1989) J. Appl. Physiol. 67: 959-962).

The potential of PGD₂ to induce inflammatory responses has been confirmed by the use of transgenic mice overexpressing human PGD₂ synthase which exhibit exaggerated eosinophilic lung inflammation and Th2 cytokine production in response to antigen (Fujitani et al, (2002) J. Immunol. 168: 443-449).

The first receptor specific for PGD₂ to be discovered was the DP₁ receptor which is linked to elevation of the intracellular levels of cAMP. However, PGD₂ is thought to mediate much of its proinflammatory activity through interaction with a G protein-coupled receptor termed CRTH2 (chemoattractant receptor-homologous molecule expressed on Th2 cells) which is expressed by Th2 lymphocytes, eosinophils and basophils (Hirai et al, (2001) J. Exp. Med. 193: 255-261, and EP0851030 and EP-A-1211513 and Bauer et al, EP-A-1170594). It seems clear that the effect of PGD₂ on the activation of Th2 lymphocytes and eosinophils is mediated through CRTH2 since the selective CRTH2 agonists 13,14 dihydro-15-keto-PGD₂ (DK-PGD₂) and 15R-methyl-PGD₂ can elicit this response and the effects of PGD₂ are blocked by an anti-CRTH2 antibody (Hirai et al, 2001; Monneret et al, (2003) J. Pharmacol. Exp. Ther. 304: 349-355). In contrast, the selective DP agonist BW245C does not promote migration of Th2 lymphocytes or eosinophils (Hirai et al, 2001; Gervais et al, (2001) J. Allergy Clin. Immunol. 108: 982-988). Based on this evidence, antagonising PGD₂ at the CRTH2 receptor is an attractive approach to treat the inflammatory component of Th2-dependent allergic diseases such as asthma (including allergic asthma), food allergies, acute and chronic urticaria, perennial allergic rhinitis, seasonal allergic rhinitis, atopic dermatitis, contact hypersensitivity (including contact dermatitis) and conjunctivitis, especially allergic conjunctivitis.

Documents which discuss the use of CRTH2 antagonists for the treatment of Th2-dependent allergic diseases include WO-A-03/066046, WO-A-03/066047, WO-A-03/097042, WO-A-03/097598, WO-A-03/101981, WO-A-03/101961, WO-A-2004/007451, WO-A-2005/019171, WO-A-2005/094816, WO-A-2005/044260, WO-A-2005/040112, WO-A-2005/040114, WO2006/095183, and WO2008/012511.

Other compounds which are combined CRTH2 and TP receptor antagonists are known and an example of such a compound is ramatroban, which has been shown to reduce allergic inflammation in the guinea pig nasal mucosa (Narita et al, Int Arch Allergy Immunol 1996; 109:161-166), mouse airways (Nagai et al, Prostaglandins 1995; 50:75-87) and mouse skin (Takeshita et al, Int. Immunol. 2004; 16:947-959). Ramatroban has also shown to reduce symptoms of perennial allergic rhinitis in human subjects (Terada et al, Allergol. Int. 1998; 47: 59-67). Analogues of ramatroban that retain CRTH2 antagonist activity, but which are inactive on TP, are effective in reducing airway eosinophilia and mucus cell hyperplasia in a model of allergic asthma in mice (Uller et al, Respir Res 2007; 8:16). Uller et al concluded that the efficacy in the allergic asthma model resulted from blockade of CRTH2 receptors rather than inhibition of TP.

### BRIEF SUMMARY OF THE INVENTION

One aspect of the invention is to provide a method of preventing, treating, or ameliorating an allergic condition in an individual, comprising administering to the individual a therapeutically effective amount of a compound of general formula (I):

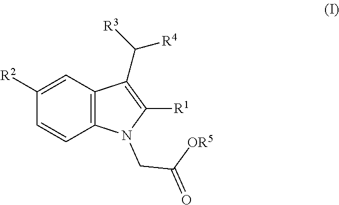

wherein
R¹ is C₁-C₆ alkyl;
R² is halogen;
R³ is aryl or heteroaryl optionally substituted with one or more substituents selected from halo, OH, CN, R⁶, COR⁶, CH₂R⁶, OR⁶, SR⁶, SO₂R⁶, or SO₂YR⁶;
R⁶ is C₁-C₆ alkyl, C₃-C₈ cycloalkyl, heterocyclyl, aryl, or heteroaryl, any of which may optionally be substituted with one or more substituents selected from halo, OH, CN, NO₂, C₁-C₆ alkyl, or O(C₁-C₆ alkyl); and
Y is NH or a straight or branched C₁-C₄ alkylene chain;
   R⁴ is H or C₁-C₄ alkyl; and
   R⁵ is hydrogen, C₁-C₆ alkyl, aryl, (CH₂)ₘOC(═O)C₁-C₆alkyl, ((CH₂)ₘO)ₙCH₂CH₂X, (CH₂)ₘN(R⁷)₂, or CH((CH₂)ₘO(C═O)R⁸)₂;
m is 1 or 2;
n is 1-4;
X is OR⁷ or N(R⁷)₂;
R⁷ is hydrogen or methyl;
R⁸ is C₁-C₁₈ alkyl;
   or a pharmaceutically acceptable salt, hydrate, solvate, or complex thereof;
   wherein the therapeutically effective amount is administered for at least one treatment cycle comprising:
(a) a first period during which the compound of general formula (I) is administered to the patient; and
(b) a second period of at least seven days during which the compound of general formula (I) is administered to the patient in a reduced amount;
   wherein the compound of general formula (I) retains at least 50% effectiveness during the second period of the treatment cycle.

In one embodiment, R⁵ of general formula (I) is hydrogen.

In one embodiment, R⁵ of general formula (I) is C₁-C₆ alkyl, aryl, (CH₂)ₘOC(═O)C₁-C₆alkyl, ((CH₂)ₘO)ₙCH₂CH₂X, (CH₂)ₘN(R⁷)₂, or CH((CH₂)ₘO(C═O)R⁸)₂.

In one embodiment, R¹ of general formula (I) is C₁-C₄ alkyl; R² is fluoro; R³ is optionally substituted and is quinoline, quinoxaline, isoquinoline, thiazole, phenyl, naphthalene, thiophene, pyrrole, or pyridine; and R⁴ is H or methyl.

In one embodiment, R⁴ of general formula (I) is H.

In one embodiment, R³ of general formula (I) is optionally substituted and is quinoline, isoquinoline, phenyl, naphthalene, thiophene, pyrrole, or pyridine.

In one embodiment, R³ of general formula (I) is quinoline or isoquinoline, wherein the quinoline or isoquinoline is unsubstituted or substituted with one or more halo substituents.

In one embodiment, R³ of general formula (I) is optionally substituted with one or more substituents and is phenyl, naphthalene, thiophene, pyrrole, or pyridine, wherein the one or more substituents are OR⁶, SO₂R⁶ or SO₂YR⁶.

In one embodiment, R⁶ of general formula (I) is optionally substituted and is C₁-C₆ alkyl, a 4- to 6-membered cycloalkyl group, a 5- or 6-membered heterocyclyl group, or phenyl.

In one embodiment, R³ of general formula (I) is a 3-pyridyl moiety.

In one embodiment, R³ of general formula (I) is substituted with SO₂YR⁶, wherein Y is a CH₂ moiety.

In one embodiment, R³ of general formula (I) is substituted with SO₂R⁶ or SO₂YR⁶, wherein the R⁶ group is unsubstituted or substituted with one or more substituents selected from methyl and halo.

In one embodiment, R³ of general formula (I) is substituted with OR⁶, wherein the R⁶ group is unsubstituted or substituted with one or more substituents selected from the group consisting of halo, cyano, C₁-C₄ alkyl, and O(C₁-C₄ alkyl).

In one embodiment, the compound of general formula (I) is:
{3-[1-(4-Chloro-phenyl)-ethyl]-5-fluoro-2-methyl-indol-1-yl}-acetic acid;
{5-Fluoro-2-methyl-3-[1-(4-trifluoromethyl-phenyl)-ethyl]-indol-1-yl}-acetic acid;
{3-[1-(4-tert-Butyl-phenyl)-ethyl]-5-fluoro-2-methyl-indol-1-yl}-acetic acid;
{5-Fluoro-3-[1-(4-methanesulfonyl-phenyl)-ethyl]-2-methyl-indol-1-yl}-acetic acid;
[5-Fluoro-2-methyl-3-(1-naphthalen-2-yl-ethyl)-indol-1-yl]-acetic acid;
(5-Fluoro-2-methyl-3-quinolin-2-ylmethyl-indol-1-yl)-acetic acid;
(5-Fluoro-2-methyl-3-naphthalen-2-ylmethyl-indol-1-yl)-acetic acid;
[5-Fluoro-3-(8-hydroxyquinolin-2-ylmethyl)-2-methyl-indol-1-yl]-acetic acid;
[5-Fluoro-2-methyl-3-(quinoxalin-2-ylmethyl)indol-1-yl]-acetic acid;
[5-Fluoro-3-(4-methoxy-benzyl)-2-methyl-indol-1-yl]-acetic acid;
[5-Fluoro-2-methyl-3-(1,3-thiazol-2-ylmethyl)indol-1-yl]-acetic acid;
[3-(4-Chloro-benzyl)-5-fluoro-2-methyl-indol-1-yl]-acetic acid;
[5-Fluoro-2-methyl-3-(4-trifluoromethyl-benzyl)-indol-1-yl]-acetic acid;
[5-Fluoro-2-methyl-3-(4-tert-butyl-benzyl)-indol-1-yl]-acetic acid;
{5-Fluoro-2-methyl-3-[(4-phenylphenyl)methyl]indol-1-yl}-acetic acid;
[5-Fluoro-3-(4-methanesulfonyl-benzyl)-2-methyl-indol-1-yl]-acetic acid;
{5-Fluoro-3-[(6-fluoroquinolin-2-yl)methyl]-2-methylindol-1-yl}-acetic acid;
(2-Methyl-3-quinolin-2-ylmethyl-indol-1-yl)-acetic acid;
(5-Chloro-2-methyl-3-quinolin-2-ylmethyl-indol-1-yl)-acetic acid;
(3-{[1-(Benzenesulfonyl)pyrrol-2-yl]methyl}-5-fluoro-2-methylindol-1-yl)-acetic acid;
[5-Fluoro-2-methyl-3-({1-[(4-methylbenzene)sulfonyl]pyrrol-2-yl}methyl)indol-1-yl]-acetic acid;
[3-({1-[(2,4-Difluorobenzene)sulfonyl]pyrrol-2-yl}methyl)-5-fluoro-2-methylindol-1-yl]-acetic acid;
(3-{[2-(Benzenesulfonyl)phenyl]methyl}-5-fluoro-2-methylindol-1-yl)-acetic acid;
[3-({2-[(4-Chlorobenzene)sulfonyl]phenyl}methyl)-5-fluoro-2-methylindol-1-yl]-acetic acid;
[5-Fluoro-3-({2-[(4-fluorobenzene)sulfonyl]phenyl}methyl)-2-methylindol-1-yl]-acetic acid;
(3-{[2-(Benzenesulfonyl)pyridin-3-yl]methyl}-5-fluoro-2-methylindol-1-yl)-acetic acid;
[5-Fluoro-3-({2-[(4-fluorobenzene)sulfonyl]pyridin-3-yl}methyl)-2-methylindol-1-yl]-acetic acid;
[3-({2-[(4-Chlorobenzene)sulfonyl]pyridin-3-yl}methyl)-5-fluoro-2-methylindol-1-yl]acetic acid;
2-(3-(4-(Benzylsulfonyl)benzyl)-5-fluoro-2-methyl-indol-1-yl)-acetic acid;
2-(3-(4-(4-Chlorobenzylsulfonyl)benzyl)-5-fluoro-2-methyl-indol-1-yl)-acetic acid;
2-(3-(3-(Benzylsulfonyl)benzyl)-5-fluoro-2-methyl-indol-1-yl)-acetic acid;
2-(5-Fluoro-3-(3-(4-fluorobenzylsulfonyl)benzyl)-2-methyl-indol-1-yl)-acetic acid;
2-(3-(2-(Benzylsulfonyl)benzyl)-5-fluoro-2-methyl-indol-1-yl)-acetic acid;
2-(3-(4-(4-Fluorobenzylsulfonyl)benzyl)-5-fluoro-2-methyl-indol-1-yl)-acetic acid;
2-(3-(2-(Cyclohexylsulfonyl)benzyl)-5-fluoro-2-methyl-indol-1-yl)-acetic acid;
2-(5-Fluoro-2-methyl-3-(2-(piperidin-1-ylsulfonyl)benzyl)-indol-1-yl)-acetic acid;
2-(3-(2-(Cyclopentylsulfonyl)benzyl)-5-fluoro-2-methyl-indol-1-yl)-acetic acid;
2-(5-Fluoro-2-methyl-3-(3-(piperidin-1-ylsulfonyl)benzyl)-indol-1-yl)-acetic acid;
2-(5-Fluoro-2-methyl-3-(2-(pyrrolidin-1-ylsulfonyl)benzyl)-indol-1-yl)-acetic acid;
2-(3-(4-(Cyclohexylsulfonyl)benzyl)-5-fluoro-2-methyl-indol-1-yl)-acetic acid;
2-(3-(4-(Cyclopentylsulfonyl)benzyl)-5-fluoro-2-methyl-indol-1-yl)-acetic acid;
2-(3-(2-(Cyclobutylsulfonyl)benzyl)-5-fluoro-2-methyl-indol-1-yl)-acetic acid;
2-(5-Fluoro-2-methyl-3-(3-(pyrrolidin-1-ylsulfonyl)benzyl)-indol-1-yl)-acetic acid;
2-(5-Fluoro-2-methyl-3-(4-(piperidin-1-ylsulfonyl)benzyl)-indol-1-yl)-acetic acid;
[5-Fluoro-2-methyl-3-(2-phenoxybenzyl)-indol-1-yl]-acetic acid;
[5-Fluoro-2-methyl-3-(2-(4-methoxyphenoxy)benzyl)-indol-1-yl]-acetic acid;
[5-Fluoro-2-methyl-3-(2-(4-methylphenoxy)benzyl)-indol-1-yl]-acetic acid;
[5-Fluoro-2-methyl-3-(2-(2,4-dichlorophenoxy)benzyl)-indol-1-yl]-acetic acid;
[5-Fluoro-2-methyl-3-(2-(4-fluorophenoxy)benzyl)-indol-1-yl]-acetic acid;
[5-Fluoro-2-methyl-3-(2-(3,4-difluorophenoxy)benzyl)-indol-1-yl]-acetic acid;
[5-Fluoro-2-methyl-3-(2-(4-cyanophenoxy)benzyl)-indol-1-yl]-acetic acid;
[5-Fluoro-2-methyl-3-(2-(4-chlorophenoxy)benzyl)-indol-1-yl]-acetic acid;
[5-Fluoro-2-methyl-3-(2-(2-cyanophenoxy)benzyl)-indol-1-yl]-acetic acid;
(5-Fluoro-2-methyl-3-{[2-(4-methylphenoxy)pyridin-3-yl]methyl}indol-1-yl)-acetic acid;
{5-Fluoro-3-[(3-methanesulfonylnaphthalen-2-yl)methyl]-2-methylindol-1-yl}-acetic acid;
{5-Fluoro-3-[(1-methanesulfonylnaphthalen-2-yl)methyl]-2-methylindol-1-yl}-acetic acid;
{5-Fluoro-3-[(6-methanesulfonylnaphthalen-2-yl)methyl]-2-methylindol-1-yl}-acetic acid;
[5-Fluoro-2-methyl-3-(quinolin-3-ylmethyl)indol-1-yl]-acetic acid;
[5-Fluoro-2-methyl-3-(quinoxalin-6-ylmethyl)indol-1-yl]-acetic acid;
[5-Fluoro-2-methyl-3-(quinolin-7-ylmethyl)indol-1-yl]-acetic acid;
{5-Fluoro-3-[(6-methanesulfonylquinolin-2-yl)methyl]-2-methylindol-1-yl}-acetic acid;
{5-Fluoro-3-[(4-methanesulfonylquinolin-2-yl)methyl]-2-methylindol-1-yl}-acetic acid;
(5-Fluoro-2-methyl-3-{pyrazolo[1,5-a]pyridin-3-ylmethyl}indol-1-yl)-acetic acid;
(5-Fluoro-3-{imidazo[1,2-a]pyridin-2-ylmethyl}-2-methylindol-1-yl)-acetic acid;
(5-Fluoro-2-methyl-3-{[2-(methylsulfanyl)phenyl]methyl}indol-1-yl)-acetic acid;
(5-Fluoro-2-methyl-3-{[3-(methylsulfanyl)phenyl]methyl}indol-1-yl)-acetic acid;
(5-Fluoro-2-methyl-3-{[4-(ethylsulfanyl)phenyl]methyl}indol-1-yl)-acetic acid;
(3-{[4-(Ethylsulfanyl)phenyl]methyl}-5-fluoro-2-methylindol-1-yl)-acetic acid;
(5-Fluoro-2-methyl-3-{[4-(n-propylsulfanyl)phenyl]methyl}indol-1-yl)-acetic acid;
(5-Fluoro-2-methyl-3-{[4-(i-propylsulfanyl)phenyl]methyl}indol-1-yl)-acetic acid;
(5-Fluoro-2-methyl-3-{[4-(t-butylsulfanyl)phenyl]methyl}indol-1-yl)-acetic acid;
(5-Fluoro-2-methyl-3-{[4-(pentan-3-ylsulfanyl)phenyl]methyl}indol-1-yl)-acetic acid;
[3-({4-[(Cyclopropylmethyl)sulfanyl]phenyl}methyl)-5-fluoro-2-methylindol-1-yl]-acetic acid;
{3-[(4,4-Dimethyl-2,3-dihydro-1-benzothiopyran-6-yl)methyl]-5-fluoro-2-methylindol-1-yl}-acetic acid;
(3-{[2-(Ethanesulfonyl)phenyl]methyl}-5-fluoro-2-methylindol-1-yl)-acetic acid;
(5-Fluoro-2-methyl-3-{[2-(propane-1-sulfonyl)phenyl]methyl}indol-1-yl)-acetic acid;
(5-Fluoro-2-methyl-3-{[2-(propane-2-sulfonyl)phenyl]methyl}indol-1-yl)-acetic acid;
(3-{[2-(Butane-1-sulfonyl)phenyl]methyl}-5-fluoro-2-methylindol-1-yl)-acetic acid;
(3-{[2-(Butane-2-sulfonyl)phenyl]methyl}-5-fluoro-2-methylindol-1-yl)-acetic acid;
(5-Fluoro-2-methyl-3-{[2-(2-methylpropane-2-sulfonyl)phenyl]methyl}indol-1-yl)-acetic acid;
(5-Fluoro-2-methyl-3-{[2-(pentane-1-sulfonyl)phenyl]methyl}indol-1-yl)-acetic acid;
(3-{[2-(Cyclopropylmethane)sulfonylphenyl]methyl}-5-fluoro-2-methylindol-1-yl)-acetic acid;
(5-Fluoro-2-methyl-3-{[2-(propylsulfamoyl)phenyl]methyl}indol-1-yl)-acetic acid;
(3-{[2-(Butylsulfamoyl)phenyl]methyl}-5-fluoro-2-methylindol-1-yl)-acetic acid;
(5-Fluoro-2-methyl-3-{[3-(propylsulfamoyl)phenyl]methyl}indol-1-yl)-acetic acid;
(3-{[3-(Butylsulfamoyl)phenyl]methyl}-5-fluoro-2-methylindol-1-yl)-acetic acid;
(5-Fluoro-2-methyl-3-{[4-(trifluoromethane)sulfonylphenyl]methyl}indol-1-yl)-acetic acid;
(3-{[4-(Ethanesulfonyl)phenyl]methyl}-5-fluoro-2-methylindol-1-yl)-acetic acid;
(5-Fluoro-2-methyl-3-{[4-(propane-1-sulfonyl)phenyl]methyl}indol-1-yl)-acetic acid;
(5-Fluoro-2-methyl-3-{[4-(propane-2-sulfonyl)phenyl]methyl}indol-1-yl)-acetic acid;
(3-{[4-(Butane-1-sulfonyl)phenyl]methyl}-5-fluoro-2-methylindol-1-yl)-acetic acid;
(5-Fluoro-2-methyl-3-{[4-(2-methylpropane-2-sulfonyl)phenyl]methyl}indol-1-yl)-acetic acid;
(5-Fluoro-2-methyl-3-{[4-(pentane-1-sulfonyl)phenyl]methyl}indol-1-yl)-acetic acid;
(5-Fluoro-2-methyl-3-{[4-(pentan-3-ylsulfonyl)phenyl]methyl}indol-1-yl)-acetic acid;
[3-({4-[(Cyclopropylmethyl)sulfonyl]phenyl}methyl)-5-fluoro-2-methylindol-1-yl]-acetic acid;
(5-Fluoro-2-methyl-3-{[4-(propylsulfamoyl)phenyl]methyl}indol-1-yl)-acetic acid;
(3-{[4-(Butylsulfamoyl)phenyl]methyl}-5-fluoro-2-methylindol-1-yl)-acetic acid;
(5-Fluoro-2-methyl-3-{[4-(trifluoromethoxy)phenyl]methyl}indol-1-yl)-acetic acid;
(5-Fluoro-3-{[4-methanesulfonyl-3-(trifluoromethyl)phenyl]methyl}-2-methylindol-1-yl)-acetic acid;
(5-Fluoro-3-{[4-methanesulfonyl-3-(trifluoromethoxy)phenyl]methyl}-2-methylindol-1-yl)-acetic acid;
{5-Fluoro-3-[(5-methanesulfonylthiophen-2-yl)methyl]-2-methylindol-1-yl}-acetic acid;
{3-[(4,4-dimethyl-1,1-dioxo-2,3-dihydro-1λ⁶-benzothiopyran-6-yl)methyl]-5-fluoro-2-methylindol-1-yl}-acetic acid;
[3-({1-[(4-Chlorobenzene)sulfonyl]pyrrol-2-yl}methyl)-5-fluoro-2-methylindol-1yl]-acetic acid;
[5-Fluoro-3-({1-[(4-fluorobenzene)sulfonyl]pyrrol-2-yl}methyl)-2-methylindol-1-yl]-acetic acid;
[5-Fluoro-3-({1-[(4-methoxybenzene)sulfonyl]pyrrol-2-yl}methyl)-2-methylindol-1-yl]-acetic acid;
{3-[1-(2,4-Dichloro-benzenesulfonyl)pyrrol-2-ylmethyl]-5-fluoro-2-methyl-indol-1-yl}-acetic acid;
[5-Fluoro-3-({1-[(4-methanesulfonylbenzene)sulfonyl]pyrrol-2-yl}methyl)-2-methylindol-1-yl]-acetic acid;
{5-Fluoro-2-methyl-3-[(2-phenylphenyl)methyl]indol-1-yl}-acetic acid;
(3-{[1-(Benzenesulfonyl)indol-2-yl]methyl}-5-fluoro-2-methylindol-1-yl)-acetic acid;
(3-{[2-(4-Chlorophenyl)phenyl]methyl}-5-fluoro-2-methylindol-1-yl)-acetic acid;
(5-Fluoro-2-methyl-3-{[2-(4-methylphenyl)phenyl]methyl}indol-1-yl)-acetic acid;
{5-Fluoro-2-methyl-3-[(3-phenoxyphenyl)methyl]indol-1-yl}-acetic acid;
[5-Fluoro-3-({4-[(4-fluorophenyl)carbonyl]-1-methylpyrrol-2-yl}methyl)-2-methylindol-1-yl]-acetic acid;
{5-Fluoro-2-methyl-3-[(6-{[3-(trifluoromethyl)phenyl]methyl}pyridin-3-yl)methyl]indol-1-yl}-acetic acid;
{-Fluoro-2-methyl-3-[(3-phenoxythiophen-2-yl)methyl]indol-1-yl}-acetic acid;
(3-{[2-(Benzenesulfonyl)-1,3-thiazol-5-yl]methyl}-5-fluoro-2-methylindol-1-yl)-acetic acid;
{3-[(1-Benzylpyrazol-4-yl)methyl]-5-fluoro-2-methylindol-1-yl}-acetic acid;
(3-{[5-(4-Chlorophenoxy)-1-methyl-3-(trifluoromethyl)pyrazol-4-yl]methyl}-5-fluoro-2-methylindol-1-yl)-acetic acid;
[3-({5-[(4-Chlorobenzene)sulfonyl]furan-2-yl}methyl)-5-fluoro-2-methylindol-1-yl]-acetic acid;
[3-({5-[(4-Chlorobenzene)sulfonyl]thiophen-2-yl}methyl)-5-fluoro-2-methylindol-1-yl]acetic acid;
[3-({3-[(4-Chlorobenzene)sulfonyl]thiophen-2-yl}methyl)-5-fluoro-2-methylindol-1-yl]-acetic acid;
{3-[(2-Benzylphenyl)methyl]-5-fluoro-2-methylindol-1-yl}-acetic acid;
   or the C₁-C₆ alkyl, aryl, (CH₂)ₘOC(═O)C₁-C₆alkyl, ((CH₂)ₘO)ₙCH₂CH₂X, (CH₂)ₘN(R⁷)₂, or CH((CH₂)ₘO(C═O)R⁸)₂ esters of any of the above; wherein
   m is 1 or 2;
   n is 1-4;
   X is OR⁷ or N(R⁷)₂;
   R⁷ is hydrogen or methyl;
   R⁸ is C₁-C₁₈ alkyl.

In one embodiment, the compound of general formula (I) is administered in combination with one or more additional agent which is of use in the treatment of allergic conditions.

In one embodiment, the additional agent administered in combination with the compound of general formula (I) is selected from the group consisting of:

Suplatast tosylate and similar compounds;
B₂ adrenoreceptor agonists such as metaproterenol, isoproterenol, isoprenaline, albuterol, salbutamol, formoterol, salmeterol, terbutaline, orciprenaline, bitolterol mesylate, pirbuterol and indacaterol or methylxanthanines such as theophylline and aminophylline, mast cell stabilisers such as sodium cromoglycate or muscarinic receptor antagonists such as ipratropium and tiotropium;
antihistamines, for example histamine H₁ receptor antagonists such as loratadine cetirizine, desloratadine, levocetirizine, fexofenadine, astemizole, azelastine and chlorpheniramine or H₄ receptor antagonists;
α₁ and α₂ adrenoreceptor agonists such as propylhexedrine phenylephrine, phenylpropanolamine, pseudoephedrine, naphazoline hydrochloride, oxymetazoline hydrochloride, tetrahydrozoline hydrochloride, xylometazoline hydrochloride and ethylnorepinephrine hydrochloride;
corticosteroids such as prednisone, prednisolone, flunisolide, triamcinolone acetonide, beclomethasone dipropionate, budesonide, fluticasone propionate, fluticasone furoate, mometasone furoate and ciclesonide; and
allergen immunotherapy such as Grazax.

In one embodiment, the compound of general formula (I) is administered via the oral, nasal, bronchial, or topical route.

In one embodiment, the individual experiences a reduction in symptoms of at least 50% during the first period of the treatment cycle.

In one embodiment, the compound of general formula (I) retains at least 80% effectiveness during the second period of the treatment cycle.

In one embodiment, the compound of general formula (I) is not administered during the second period of the treatment cycle.

In one embodiment, the second period of the treatment cycle is at least 28 days.

In one embodiment, the first period of the treatment cycle is from two to eight days.

In one embodiment, the first period further comprises administering the compound of general formula (I) once daily.

In one embodiment, the invention further comprises one or more additional treatment cycles.

In one embodiment, the allergic condition is selected from the group consisting of asthma, allergic asthma, food allergies, acute and chronic urticaria, perennial allergic rhinitis, seasonal allergic rhinitis, atopic dermatitis, contact hypersensitivity, contact dermatitis, conjunctivitis, and allergic conjunctivitis.

In another embodiment, the present invention provides a method for the prolonged reduction of the severity of the symptoms produced by an allergen in a patient, comprising:

(a) administering a therapeutic amount of a compound of general formula (I):

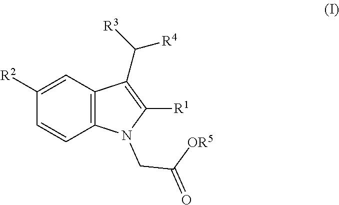

wherein
R¹ is C₁-C₆ alkyl;
R² is halogen;
R³ is aryl or heteroaryl optionally substituted with one or more substituents selected from halo, OH, CN, R⁶, COR⁶, CH₂R⁶, OR⁶, SR⁶, SO₂R⁶, or SO₂YR⁶;
R⁶ is C₁-C₆ alkyl, C₃-C₈ cycloalkyl, heterocyclyl, aryl, or heteroaryl, any of which may optionally be substituted with one or more substituents selected from halo, OH, CN, NO₂, C₁-C₆ alkyl, or O(C₁-C₆ alkyl); and
Y is NH or a straight or branched C₁-C₆ alkylene chain;
   R⁴ is H or C₁-C₄ alkyl; and
   R⁵ is hydrogen, C₁-C₆ alkyl, aryl, (CH₂)ₘOC(═O)C₁-C₆alkyl, ((CH₂)ₘO)ₙCH₂CH₂X, (CH₂)ₘN(R⁷)₂, or CH((CH₂)ₘO(C═O)R⁸)₂;
m is 1 or 2;
n is 1-4;
X is OR⁷ or N(R⁷)₂;
R⁷ is hydrogen or methyl;
R⁸ is C₁-C₁₈ alkyl;
   or a pharmaceutically acceptable salt, hydrate, solvate, or complex thereof; and
(b) ceasing administration of said compound of general formula (I);
   wherein the symptoms of the allergic condition continue to be reduced after said compound of general formula (I) is no longer in the body of the patient.

In one embodiment, the present invention provides a method for the prolonged reduction but not the prevention of the severity of the symptoms produced by an allergen in a patient.

In one embodiment, the atopic state of the patient is reduced.

In one embodiment, the symptoms of the allergic condition continue to be reduced for at least 7 days after the cessation of treatment with the compound of general formula (I).

In one embodiment, the symptoms of the allergic condition continue to be reduced for at least 35 days after ceasing administration of the compound of general formula (I).

In one embodiment, the reduction of atopic state, as measured either by a reduction in the number of cells reactive to allergens or the level of circulating IgE or the reduction in positive reaction in a skin prick allergy test is at least 30%.

In one embodiment, the reduction of atopic state, as measured either by a reduction in the number of cells reactive to allergens or the level of circulating IgE or the reduction in positive reaction in a skin prick allergy test is at least 60%.

### BRIEF DESCRIPTION OF THE FIGURES

FIG. 1 is a series of plots showing the combined symptom scores of two groups of patients with allergic rhinitis in a test comparing a CRTH2 antagonist with a placebo on Days 2 and 8 of each treatment period. FIG. 1A shows the combined nasal symptom scores for both patient groups in response to challenge with a CRTH2 antagonist, while FIG. 1B shows the combined scores for eye symptoms. The plots show that patients taking the CRTH2 antagonist show reduced nasal and eye symptoms on both Day 2 and Day 8 of the treatment period.

FIG. 2 is a series of plots similar to that of FIG. 1 but where the scores for each patient group have been shown separately. The plots also show a screening response, which is the response of the patients to allergen before the beginning of the trial. FIGS. 2A and 2B show the total nasal symptoms for patient group A compared with the screening response score for the same group of patients. FIG. 2A is plot showing the scores for nasal symptoms of patients in Group A on day 2 of each of the treatment periods while FIG. 2B shows the nasal symptom scores for the same group of patients on day 8. Patient group A was treated with CRTH2 antagonist for 8 days, then given no treatment for 3 weeks, then treated with placebo for 8 days.

FIGS. 2C and 2D are similar to FIGS. 2A and 2B but show the scores for nasal symptoms from patients from patient group B on days 2 and 8 of the treatment periods together with the screening response score for the same group of patients. Patient group B was treated with placebo for 8 days, then given no treatment for 3 weeks, then treated with CRTH2 antagonist for 8 days.

FIG. 3 is a plot showing the levels of serum IgE in asthma patients at the beginning of a study and after dosing with either a CRTH2 antagonist or placebo for a period of 4 weeks.

FIG. 4 is a plot showing the anti-apoptotic effect of PGD2 in human Th2 cells. Human Th2 cells were treated with 50 U/ml IL-2 or various concentrations of PGD₂ in the absence of IL-2 for 16 hrs. The cells were stained with Annexin V-PE/PI and then analysed by FACSArray flow cytometer.

### DETAILED DESCRIPTION OF THE INVENTION

Although there is ample evidence that CRTH2 antagonists are effective in treating allergic asthma, allergic rhinitis, allergic conjunctivitis, atopic dermatitis and other allergic conditions, the inventors have now made the surprising discovery that these compounds have an effect which is much longer lasting than previously thought and are thus useful preventing or reducing the symptoms of the allergic condition for a prolonged period of time after dosing.

Therefore, in a first aspect of the invention there is provided a compound of general formula (I):

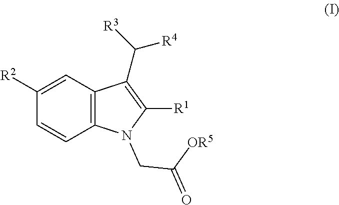

wherein
R¹ is C₁-C₆ alkyl;
R² is halogen; and
R³ is aryl or heteroaryl optionally substituted with one or more substituents selected from halo, OH, CN, R⁶, COR⁶, CH₂R⁶, OR⁶, SR⁶, SO₂R⁶ or SO₂YR⁶;
R⁶ is C₁-C₆ alkyl, C₃-C₈ cycloalkyl, heterocyclyl, aryl or heteroaryl, any of which may optionally be substituted with one or more substituents selected from halo, OH, CN, NO₂, C₁-C₆ alkyl or O(C₁-C₆ alkyl); and
Y is NH or a straight or branched C₁-C₄ alkylene chain;
   R⁴ is H or C₁-C₄ alkyl;
   R⁵ is hydrogen, C₁-C₆ alkyl, aryl, (CH₂)ₘOC(═O)C₁-C₆alkyl, ((CH₂)ₘO)ₙCH₂CH₂X, (CH₂)ₘN(R⁷)₂ or CH((CH₂)ₘO(C═O)R⁸)₂;
m is 1 or 2;
n is 1-4;
X is OR⁷ or N(R⁷)₂;
R⁷ is hydrogen or methyl;
R⁸ is C₁-C₁₈ alkyl;
   or a pharmaceutically acceptable salt, hydrate, solvate, or complex thereof;
   for the treatment of an allergic condition which is induced by the presence of an allergen to which the patient is sensitive, wherein the treatment comprises at least one treatment cycle which comprises:
   a. a first period during which the compound of general formula (I) is administered to the patient; and
   b. a second period of at least seven days during which the compound of general formula (I) is administered to the patient in a reduced amount;
   wherein the allergen is present or suspected to be present throughout the treatment and wherein the compound of general formula (I) retains at least 50% of its effectiveness during the second period of the treatment cycle.

This is particularly advantageous for conditions such as allergic rhinitis, allergic conjunctivitis or allergic asthma where during the period in which the patient suffers symptoms, medication conventionally has to be taken at least once a day, and often more frequently, in order to suppress those symptoms. In the present invention, treatment is taken as normal during the first period of the treatment cycle but during the second period a greatly reduced dosage of the compound of general formula (I) can be taken and in some cases, the dosage may be reduced to zero during the second period of the treatment cycle. This means that in the case of a condition such as allergic rhinitis where the hay fever season lasts for several weeks or even months, it may only be necessary for the patient to take medication for a small proportion of that season.

The invention arises from observations made in the course of a double blind crossover study of a CRTH2 antagonist of general formula (I) in human subjects with allergic rhinitis. The study compared two groups of patients. The first patient group was treated daily with the CRTH2 antagonist for 8 consecutive days, then had no treatment for 3 weeks, then had daily treatment with placebo for 8 consecutive days. The second patient group was treated with placebo for 8 consecutive days, then had no treatment for 3 weeks, then had daily treatment with the CRTH2 antagonist of general formula (I) for 8 consecutive days. The study showed that in the first treatment period, patients treated with the CRTH2 antagonist of general formula (I) displayed fewer symptoms of allergic rhinitis than patients treated with a placebo and this result was expected by the inventors. Surprisingly, however, in the second treatment period, the first patient group, which was being treated with placebo, still retained a reduction in symptoms, the results being comparable with those of the second patient group which was being treated with the CRTH2 antagonist of general formula (I) at that time. These patients also displayed fewer symptoms than they had displayed in an initial screening before treatment with either CRTH2 antagonist or placebo.

It therefore appears that CRTH2 antagonists such as the compounds of general formula (I) as defined above have a prolonged effect on the allergic response and that, therefore, these compounds are of use in a pulsed method for the treatment of an allergic condition which is induced by the presence of an allergen to which the patient is sensitive, the method comprising administering to a patient over at least one treatment cycle a CRTH2 antagonist compound of general formula (I), wherein the said treatment cycle comprises:

a. a first period during which a compound of general formula (I) is administered to the patient; and
b. a second period of at least seven days during which the compound of general formula (I) is administered to the patient in a reduced amount;
wherein the allergen is present or suspected to be present throughout the treatment and wherein the compound of general formula (I) retains at least 50% of its effectiveness during the second period of the treatment cycle.

The invention also provides the use of a CRTH2 antagonist compound of general formula (I) in the preparation of an agent for the treatment of an allergic condition which is induced by the presence of an allergen to which the patient is sensitive, wherein the treatment comprises at least one treatment cycle which comprises:

a. a first period during which the compound of general formula (I) is administered to the patient; and
b. a second period of at least seven days during which the compound of general formula (I) is administered to the patient in a reduced amount;
wherein the allergen is present or suspected to be present throughout the treatment and wherein the wherein the compound of general formula (I) retains at least 50% of its effectiveness during the second period of the treatment cycle.

The reason for the prolonged effect of CRTH2 antagonist compounds such as the compounds of general formula (I) in the treatment of allergic conditions is not completely clear but the inventors speculate that it may be related to a previously unknown effect of CRTH2 antagonists on Th2 cells.

The inventors have discovered that PGD₂ plays an important role to play in Th2 cell survival by preventing the apoptosis of Th2 cells and their subsequent clearance from the allergic tissue. In addition, they have discovered that CRTH2 antagonists are able to block the effect of CRTH2 and induce apoptosis of Th2 cells.

The inventors believe that it is possible that the prolonged effect may be a result of the effect of CRTH2 antagonists on apoptosis of Th2 cells.

The inventors also, when conducting a clinical trial to demonstrate the effectiveness of the compounds in treating the symptoms of these conditions, made the surprising discovery that they are able to desensitise the immune system to allergens, thus preventing or reducing the symptoms of the allergic condition for a prolonged period of time.

Therefore, in a second aspect of the invention there is provided a compound of general formula (I):

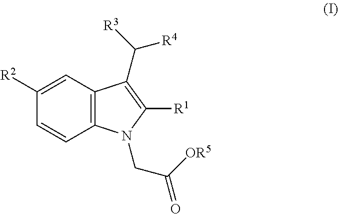

wherein
R¹ is C₁-C₆ alkyl;
R² is halogen; and
R³ is aryl or heteroaryl optionally substituted with one or more substituents selected from halo, OH, CN, R⁶, COR⁶, CH₂R⁶, OR⁶, SR⁶, SO₂R⁶ or SO₂YR⁶;
R⁶ is C₁-C₆ alkyl, C₃-C₈ cycloalkyl, heterocyclyl, aryl or heteroaryl, any of which may optionally be substituted with one or more substituents selected from halo, OH, CN, NO₂, C₁-C₆ alkyl or O(C₁-C₆ alkyl); and
Y is NH or a straight or branched C₁-C₄ alkylene chain;
   R⁴ is H or C₁-C₄ alkyl;
   R⁵ is hydrogen, C₁-C₆ alkyl, aryl, (CH₂)ₘOC(═O)C₁-C₆alkyl, ((CH₂)ₘO)ₙCH₂CH₂X, (CH₂)ₘN(R⁷)₂ or CH((CH₂)ₘO(C═O)R⁸)₂;
m is 1 or 2;
n is 1-4;
X is OR⁷ or N(R⁷)₂;
R⁷ is hydrogen or methyl;
R⁸ is C₁-C₁₈ alkyl;
   or a pharmaceutically acceptable salt, hydrate, solvate, or complex thereof;
   for use in desensitising the immune system of a subject to one or more allergens responsible for an allergic condition.

This is most surprising as, conventionally, in order to desensitise a subject, it has been necessary to administer to a subject small doses of each allergen or of peptides derived from each allergen to which the subject is sensitive. In contrast, the present inventors have discovered that the administration of a CRTH2 antagonist compound can desensitise a subject to allergy in a way which is not allergen-specific.

The present inventors postulated that the observations from the double blind crossover study could also be a result of the desensitisation of the patients to the allergic response and have confirmed this by measuring the effect of the compounds of general formula (I) on circulating levels of IgE in asthma patients. Furthermore, the reduction in IgE engendered by the compounds of general formula is not determined by the specificity of the IgE and therefore the compounds of general formula (I) are effective in desensitising any patient to any allergen. This is a great advantage as it overcomes the problems of having to tailor desensitisation treatment to individual patients.

The reason for the occurrence of the desensitising effect is not completely clear but the inventors speculate that it may be related to a previously unknown effect of CRTH2 antagonists on Th2 cells.

The inventors believe that it is possible that the desensitising effect may be a result of the effect of CRTH2 antagonists on apoptosis of Th2 cells.

It is known that Th2 lymphocytes take part in the cascade leading to an allergic reaction as described by various authors, including Pettipher et al, Nature Reviews, 6 (April 2007), 313-325. The immune response in allergy begins with the presentation of allergens to antigen presenting cells which internalise the antigens and cause them to be expressed on the cell surface. This initiates a cascade of events leading to the production of IgE, which binds to high affinity receptors on mast cells leaving its allergen-specific site available for interaction with allergen. Upon re-exposure to the same allergen, cross-linking of mast cell-bound IgE initiates the production of a number of factors, including PGD₂ by the mast cells, which in turn leads to the recruitment and activation of Th2 cells via the interaction of PGD₂ with the CRTH2 receptor. The activated Th2 cells produce various cytokines including IL4, IL5 and IL13. IL4, in turn, causes the production of further IgE by B cells.

The inventors have postulated that the apoptotic effect of CRTH2 antagonists on Th2 lymphocytes interrupts the cascade by promoting apoptosis and clearance of Th2 cells which leads to a reduction in the levels of the Th2 cytokines IL4, IL5 or IL13. The reduction in IL4 and IL13 production would lead to a decrease in the production of IgE by the B-lymphocytes. Therefore it appears that the immune system would not be able to respond to an allergen by increased production of IgE. This would lead to a significantly decreased immune response to an allergen until such time as the Th2 cell population is replenished.

Also, the reduction in IL5 would lead to reduced eosinophil migration and the reduction in IL13 to reduced mucus production. This combination of factors would also reduce the symptoms of allergy.

However, it should be noted that the long lasting effect of CRTH2 antagonists such as the compounds of general formula (I) in reducing the symptoms of an allergic condition has been shown to be present in vivo, even if this rationalisation proves to be incorrect. It should also be noted that the desensitisation effect has been shown to be present in vivo in human patients even if this rationalisation proves to be incorrect.

In the present specification “C₁-C₆ alkyl” refers to a straight or branched saturated hydrocarbon chain having one to six carbon atoms and optionally substituted with one or more halo substituents or with one or more C₃-C₇ cycloalkyl groups. Examples include methyl, ethyl, n-propyl, isopropyl, t-butyl, n-hexyl, trifluoromethyl, 2-chloroethyl, methylenecyclopropyl, methylenecyclobutyl, methylenecyclobutyl and methylenecyclopentyl.

“C₁-C₄ alkyl” and “C₁-C₁₈ alkyl” have similar meanings except that they contain from one to four and from one to eighteen carbon atoms respectively. C₃-C₇ cycloalkyl refers to a saturated 3 to 7 membered carbocyclic ring. Examples of such groups include cyclopropyl, cyclobutyl, cyclopentyl and cyclohexyl.

The term “C₁-C₄ alkylene” in the context of the present specification refers to a disubstituted straight or branched saturated hydrocarbon chain having one to four carbon atoms.

In the present specification, “halo” refers to fluoro, chloro, bromo or iodo.

The term “aryl” in the context of the present specification refers to an aromatic ring system having from 5 to 14 ring carbon atoms and containing up to three rings. Examples of aryl groups are benzene and naphthalene.

The term “heteroaryl” in the context of the specification refers to a ring system with aromatic character having from 5 to 14 ring atoms, at least one of which is a heteroatom selected from N, O and S, and containing up to three rings. Where a heteroaryl group contains more than one ring, not all rings must be fully aromatic in character. Rings which are not fully aromatic may be substituted with one or more oxo groups. Examples of heteroaryl groups include pyrrole, thiophene, thiazole, pyridine, pyrimidine, indole, benzofuran, benzimidazole, tetrahydroquinoline, indoline, quinoline, isoquinoline, quinoxaline, imidazo[1,2-a]pyridine, pyrazolo[1,5-a]pyridine, 2,3-dihydro-1-benzothiopyrane and 2,3-dihydro-1λ⁶-benzothiopyran-1,1-dione.

The term “heterocyclyl” in the context of the specification refers to a saturated ring system having from 4 to 8 ring atoms, at least one of which is a heteroatom selected from N, O and S and which may be optionally substituted by one or more oxo groups. Examples of heterocyclyl groups include azetidinyl, piperidinyl; tetrahydrofuranyl, tetrahydropyranyl, dioxanyl, thiomorpholinyl, 1,1-dioxo-1λ₆-thiomorpholinyl, morpholinyl, pyrrolyl, piperizinyl, azepanyl, 1,4-diazepanyl, 1,4-oxazepanyl and azocanyl.

Appropriate pharmaceutically and veterinarily acceptable salts of the compounds of general formula (I) include basic addition salts such as sodium, potassium, calcium, aluminium, zinc, magnesium and other metal salts as well as choline, diethanolamine, ethanolamine, ethyl diamine, megulmine and other well known basic addition salts as summarised in J. Med. Chem., 50, 6665-6672 (2007) and/or known to those skilled in the art.

Where appropriate, pharmaceutically or veterinarily acceptable salts may also include salts of organic acids, especially carboxylic acids, including but not limited to acetate, trifluoroacetate, lactate, gluconate, citrate, tartrate, maleate, malate, pantothenate, adipate, alginate, aspartate, benzoate, butyrate, digluconate, cyclopentanate, glucoheptanate, glycerophosphate, oxalate, heptanoate, hexanoate, fumarate, nicotinate, pamoate, pectinate, 3-phenylpropionate, picrate, pivalate, proprionate, tartrate, lactobionate, pivolate, camphorate, undecanoate and succinate, organic sulfonic acids such as methanesulfonate, ethanesulfonate, 2-hydroxyethane sulfonate, camphorsulfonate, 2-naphthalenesulfonate, benzenesulfonate, p-chlorobenzenesulfonate and p-toluenesulfonate; and inorganic acids such as hydrochloride, hydrobromide, hydroiodide, sulfate, bisulfate, hemisulfate, thiocyanate, persulfate, phosphoric and sulfonic acids.

Salts which are not pharmaceutically or veterinarily acceptable may still be valuable as intermediates.

The compounds of general formula (I) in which R⁵ is hydrogen are active as CRTH2 antagonists.

Prodrugs are any covalently bonded compounds which release the active parent drug according to general formula (I) in vivo. Examples of prodrugs include the compounds of general formula (I) in which R⁵ is C₁-C₆ alkyl, aryl, (CH₂)ₘOC(═O)C₁-C₆alkyl, ((CH₂)ₘO)ₙCH₂CH₂X, (CH₂)ₘN(R⁷)₂ or CH((CH₂)ₘO(C═O)R⁸)₂; where
m is 1 or 2;
n is 1-4;
X is OR⁷ or N(R⁷)₂;
R⁷ is hydrogen or methyl; and
R⁸ is C₁-C₁₈ alkyl.

If a chiral centre or another form of isomeric centre is present in a compound of the present invention, all forms of such isomer or isomers, including enantiomers and diastereoisomers, are intended to be covered herein. Compounds of the invention containing a chiral centre may be used as a racemic mixture, an enantiomerically enriched mixture, or the racemic mixture may be separated using well-known techniques and an individual enantiomer may be used alone.

The term “administered in combination with” refers to the co-administration of a CRTH2 antagonist with another drug wherein the administration may be simultaneous, sequential or separate.

In the context of the present invention, the term “allergic condition” refers to a condition in which the atopic state of a patient is increased above that of subject who does not have an allergic condition. This may be characterised by the presence in the circulation or tissues of a patient of increased numbers of cells reactive to allergens, for example mast cells, basophils, Th2 cells, dendritic cells and B cells. A further characteristic of patients with atopy may be the presence of IgE which binds specifically to an allergen to which the patient is sensitive. The IgE may be circulating or bound, for example to the surface of mast cells and basophils.

References herein to a patient being “sensitive” to an allergen mean that when the patient is exposed to the allergen, an allergic response is induced such that the patient will display symptoms of inflammation arising from the production of substances such as histamine, prostaglandin D₂ and tryptase by mast cells and basophils when the allergen binds to IgE expressed on their surfaces.

References to “the treatment of an allergic condition” refer to the reduction or removal of the inflammatory symptoms which arise from the exposure of a patient to an allergen to which the patient is sensitive.

References to “desensitisation” and “desensitising” the immune system to one or more allergens refer to the reduction in the atopic state of the patient. This may be characterised by a reduction in the numbers of cells reactive to allergens in the tissues and the blood of the patient which may be associated with a reduction in the levels of circulating and/or bound IgE in the patient after treatment as compared with the levels of circulating and/or bound IgE before treatment. Alternatively, the desensitisation may be characterised by a reduction in positivity in a standard pinprick allergy test. In the context of the present application, treatment refers to treatment with a CRTH2 antagonist, for example a compound of general formula (I).

The number of cells which are reactive to allergens in the tissue of a patient may be determined by obtaining a blood sample followed by detection of one or more types of cell which are reactive to allergens using any standard method. The number of various leukocyte populations such as mast cells, basophils, eosinophils, T lymphocytes and dendritic cells can be measured in biopsies of tissue such as the nasal mucosa. Detection of IgE is most easily achieved by quantitating IgE in a blood sample obtained from the patient, for example by an immunoassay method. Alternatively, a standard skin prick allergy test may be used with the reduction in positive result being a measure of reduction in atopy.

Thus, desensitisation is not the same as simply treating the symptoms of allergy. It is also not the same as preventing the symptoms of allergy by administering an agent such as an anti-histamine or a steroid before the symptoms occur. In a patient where the symptoms of allergy alone are treated, the atopic condition of the patient remains unchanged, as identified either by the numbers of inflammatory cells in the tissues of the patient or by the circulating levels of IgE, but some of the symptoms are reduced or removed for the period of time during which the drug remains in the body of the patient. Once the drug is cleared from the patient's body, the symptoms return.

Desensitisation actually changes the atopic state of the patient and removes the cause of the symptoms—i.e. it reduces the levels of cells reactive to allergen and/or IgE in the body of the patient. This results in a prevention or a reduction in the severity of the symptoms produced by an allergen, an effect which persists even when the compound of general formula (I) is no longer present in the body of the patient.

In general, the reduction in the atopic state of the patient will remain for at least 7 days after treatment with the compound of general formula (I) has ceased. Suitably, the reduction in atopic state remains for at least 14 days, more suitably at least 21 days, still more suitably at least 28 days or at least 35 days after the cessation of treatment. In the most favourable cases, the effect remains for longer than this, for example 2 to 6 months or even 12 months or more. For patients with allergic rhinitis this means that the desensitisation may last for the duration of the allergy season.

The reduction in atopic state, as measured either by a reduction in the number of cells reactive to allergens or the level of circulating IgE or the positivity of a skin prick test will be at least 30%, more usually at least 40% and, in increasing order of preference, at least 50%, at least 60%, at least 70%, at least 80% and at least 90%.

The reduction of the allergic response can also be measured in terms of a reduction in symptoms when a desensitised patient is exposed to allergen, as compared with the symptoms on exposure to the same allergen before treatment. The reduction in symptoms is preferably is at least 10% in symptoms, preferably at least 20% and with increasing order of preference, at least 30%, at least 40%, at least 50%, at least 60%, at least 70%, at least 80% and at least 90%.

This method of measuring desensitisation is by necessity subjective and may sometimes be considered to be less useful than the more objective measurement of IgE levels, numbers of allergen-responsive cells in blood or tissue or skin prick test positivity.

References herein to an antigen being present or suspected to be present throughout the treatment cycle are intended to mean that the antigen may be present in the patient's environment either constantly or intermittently and in varying amounts. For example, when patients suffer from allergic rhinitis, the pollen to which they are sensitive may be present in the environment throughout the hay fever season but the amount of pollen in the environment may vary depending on the weather, the time of day and other conditions. In some cases, a patient may suspect that the allergen is present in the environment, even if no tests are carried out to confirm this. Again, in the case of allergic rhinitis, a patient would conventionally take medication as a precautionary measure if it was suspected that pollen may be present, as might occur, for example, during the hay fever season. However, with the present invention, it would not be necessary for a patient to take a full dose of the compound of general formula (I) during the second period of the treatment cycle and often no compound of general formula (I) needs to be administered during this second period.

The term “effectiveness” as applied to the compounds of general formula (I) refers to the reduction of the allergic response in the patient. In general, during the first period of the treatment cycle, the patient will experience a reduction of at least 30% in symptoms, suitably at least 40% and with increasing order of suitability, at least 50%, at least 60%, at least 70% at least 80% and, in the most favourable cases, at least a 90% reduction in symptoms.

In the case of allergic rhinitis and conjunctivitis, assessment may be carried out, for example in a Vienna Challenge Chamber experiment, which is well known to those of skill in this art and in which patients carry out self-assessment and scoring of their symptoms on a scale of 0 to 3. Separate scores may be given for eye symptoms, nasal symptoms (including nasal obstruction, nasal itch, sneeze and rhinorrhea) and other symptoms. Although the symptom score for each patient is subjective, if a sufficient number of patients is used, the total scores are meaningful. For other allergic conditions, other clinical trials may be designed and the measurement of reduction in symptoms may either be carried out by a subjective method such as self assessment by patients or, alternatively, may be made by an objective measurement. For example, asthma symptoms may be quantified using measurements of lung function such as forced expiratory volume in one second (FEV₁) or peak expiratory flow rate (PEF) or using the Juniper quality of life scale. The severity of atopic dermatitis symptoms can be assessed using the scoring atopic dermatitis (SCORAD) or six area six sign atopic dermatitis (SASSAD) systems.

As set out above, during the second period of the treatment cycle, the compound of general formula (I) retains at least 50% of its effectiveness, but it is more suitable that the compound of general formula (I) retains at least 60% of its effectiveness and, in increasing order of suitability, 70%, 80% and 90% of its effectiveness.

As already noted, the amount of compound of general formula (I) administered to the patient during the second period of the treatment cycle is reduced compared with the amount administered during the first period. In general, the daily dose of compound of general formula (I) administered during the second period of the treatment cycle will be not greater than 50% of the daily dose administered during the first period. However, it may be less than this, for example not greater than 40%, not greater than 30%, not greater than 20% or not greater than 10% of the daily dose of compound of general formula (I) administered during the first period. In many cases administration of the CRTH2 antagonist compound will be discontinued altogether during the second period of the treatment cycle. Thus, in the second period of the treatment cycle, the daily dose with be zero and no compound of general formula (I) will be administered to the patient. This is a particularly advantageous result as period during which the medication is necessary will be considerably shorter than for conventional medications, which has positive implications both for the general well being of the patient and the cost of the treatment.

The second period of the treatment cycle lasts for at least 7 days. However, it may last for longer periods, for example at least 14 days and it has been observed that the drug may retain at least 50% of its effectiveness during second periods lasting at least 21 days, at least 28 days and, in some cases, in excess of 35 days even when the dose of compound is reduced to zero during the second period of the treatment cycle.

The first period of the treatment cycle during which the CRTH2 antagonist is administered may be chosen such that it comprises a period of days that provides the maximum biological response to the compound in the patient. In general, this period may be from two to 8 days, during which time the drug may be administered from one to three times a day. It is particularly suitable however that the CRTH2 antagonist is administered to the patient once daily as this helps to increase patient compliance.

In some cases, one treatment cycle may not be sufficient to last for the whole time over which treatment is required and therefore the treatment may include one or more further treatment cycles. This is often the case with conditions such as seasonal allergic rhinitis, where the hay fever season may last for several months.

CRTH2 antagonists such as the compounds of general formula (I) are useful in treating any allergic condition but they are particularly useful in the pulsed treatment of conditions such as asthma (including allergic asthma), food allergies, acute and chronic urticaria, perennial allergic rhinitis, seasonal allergic rhinitis, atopic dermatitis, contact hypersensitivity (including contact dermatitis) and conjunctivitis, especially allergic conjunctivitis.

CRTH2 antagonists such as the compounds of general formula (I) are capable of desensitising the immune system to one or more allergens responsible for any allergic condition but they are particularly useful in desensitising a patient to allergens responsible for asthma (including allergic asthma), food allergies, acute and chronic urticaria, perennial allergic rhinitis, seasonal allergic rhinitis, atopic dermatitis, contact hypersensitivity (including contact dermatitis) and conjunctivitis, especially allergic conjunctivitis.

The CRTH2 antagonists of general formula (I) are indole-1-acetic acid derivatives and are some of the compounds and analogues thereof described in WO2005/044260, WO2005/094816 and WO2006/095183, WO2008/012511 and UK patent application Nos 0800874.0, filed 18 Jan. 2008; 0801132.2, filed 22 Jan. 2008; 0801671.9, filed 30 Jan. 2008; 0801131.4, filed 22 Jan. 2008; 0801672.7, filed 30 Jan. 2008; and 0801674.3, filed 30 Jan. 2008. The indole-1-acetic acid derivatives of general formula (I) as defined above are described in detail in these documents.

In one embodiment of the invention, the compound of general formula (I) is a CRTH2 antagonist in which R⁵ is hydrogen.

In an alternative embodiment of the invention, the compound of general formula (I) is a prodrug for a CRTH2 antagonist and R⁵ is C₁-C₆ alkyl, aryl, (CH₂)ₘOC(═O)C₁-C₆alkyl, ((CH₂)ₘO)ₙCH₂CH₂X, (CH₂)ₘN(R⁷)₂ or CH((CH₂)ₘO(C═O)R⁸)₂; where
m is 1 or 2;
n is 1-4;
X is OR⁷ or N(R⁷)₂;
R⁷ is hydrogen or methyl; and
R⁸ is C₁-C₁₈ alkyl.

In suitable compounds of general formula (I), independently or in any combination:

R¹ is fluoro;
R² is C₁-C₄ alkyl, particularly methyl or ethyl but more especially methyl;
R⁴ is H or methyl; and
R³ is quinoline, quinoxaline, isoquinoline, thiazole, phenyl, naphthalene, thiophene, pyrrole or pyridine, any of which may optionally be substituted as set out above.

In particularly suitable compounds, R⁴ is H.

More typical R³ groups include optionally substituted quinoline, phenyl, naphthalene, thiophene, pyrrole or pyridine.

When R³ is quinoline or isoquinoline, it is suitably unsubstituted or substituted with one or more halo substituents, especially fluoro.

When R³ is phenyl, naphthalene, thiophene, pyrrole or pyridine, it may optionally have one or more substituents, with particularly suitable substituents including OR⁶, SO₂R⁶ or SO₂YR⁶; where R⁶ and Y are as defined above.

Typically, in this case, R⁶ is C₁-C₆ alkyl, a 4- to 6-membered cycloalkyl group, a 5- or 6-membered heterocyclyl group or phenyl, any of which may be substituted as defined above.

When R³ is pyridyl it is most suitably a 3-pyridyl moiety.

In more active compounds, Y, when present, is a CH₂ moiety.

When R³ is substituted with SO₂R⁶ or SO₂YR⁶, the R⁶ group is generally unsubstituted or substituted with one or more substituents chosen from methyl and halo, particularly chloro or fluoro.

When R³ is substituted with OR⁶, the R⁶ group may be unsubstituted or substituted with one or more substituents chosen from halo, cyano, C₁-C₄ alkyl and O(C₁-C₄ alkyl).

Particularly suitable compounds of general formula (I) include:
{3-[1-(4-Chloro-phenyl)-ethyl]-5-fluoro-2-methyl-indol-1-yl}-acetic acid;
{5-Fluoro-2-methyl-3-[1-(4-trifluoromethyl-phenyl)-ethyl]-indol-1-yl}-acetic acid;
{3-[1-(4-tert-Butyl-phenyl)-ethyl]-5-fluoro-2-methyl-indol-1-yl}-acetic acid;
{5-Fluoro-3-[1-(4-methanesulfonyl-phenyl)-ethyl]-2-methyl-indol-1-yl}-acetic acid;
[5-Fluoro-2-methyl-3-(1-naphthalen-2-yl-ethyl)-indol-1-yl]-acetic acid;
(5-Fluoro-2-methyl-3-quinolin-2-ylmethyl-indol-1-yl)-acetic acid;
(5-Fluoro-2-methyl-3-naphthalen-2-ylmethyl-indol-1-yl)-acetic acid;
[5-Fluoro-3-(8-hydroxyquinolin-2-ylmethyl)-2-methyl-indol-1-yl]-acetic acid;
[5-Fluoro-2-methyl-3-(quinoxalin-2-ylmethyl)indol-1-yl]-acetic acid;
[5-Fluoro-3-(4-methoxy-benzyl)-2-methyl-indol-1-yl]-acetic acid;
[5-Fluoro-2-methyl-3-(1,3-thiazol-2-ylmethyl)indol-1-yl]-acetic acid;
[3-(4-Chloro-benzyl)-5-fluoro-2-methyl-indol-1-yl]-acetic acid;
[5-Fluoro-2-methyl-3-(4-trifluoromethyl-benzyl)-indol-1-yl]-acetic acid;
[5-Fluoro-2-methyl-3-(4-tert-butyl-benzyl)-indol-1-yl]-acetic acid;
{5-Fluoro-2-methyl-3-[(4-phenylphenyl)methyl]indol-1-yl}-acetic acid;
[5-Fluoro-3-(4-methanesulfonyl-benzyl)-2-methyl-indol-1-yl]-acetic acid;
{5-Fluoro-3-[(6-fluoroquinolin-2-yl)methyl]-2-methylindol-1-yl}-acetic acid;
(2-Methyl-3-quinolin-2-ylmethyl-indol-1-yl)-acetic acid;
(5-Chloro-2-methyl-3-quinolin-2-ylmethyl-indol-1-yl)-acetic acid;
(3-{[1-(Benzenesulfonyl)pyrrol-2-yl]methyl}-5-fluoro-2-methylindol-1-yl)-acetic acid;
[5-Fluoro-2-methyl-3-({1-[(4-methylbenzene)sulfonyl]pyrrol-2-yl}methyl)indol-1-yl]-acetic acid;
[3-({1-[(2,4-Difluorobenzene)sulfonyl]pyrrol-2-yl}methyl)-5-fluoro-2-methylindol-1-yl]-acetic acid;
(3-{[2-(Benzenesulfonyl)phenyl]methyl}-5-fluoro-2-methylindol-1-yl)-acetic acid;
[3-({2-[(4-Chlorobenzene)sulfonyl]phenyl}methyl)-5-fluoro-2-methylindol-1-yl]-acetic acid;
[5-Fluoro-3-({2-[(4-fluorobenzene)sulfonyl]phenyl}methyl)-2-methylindol-1-yl]-acetic acid;
(3-{[2-(Benzenesulfonyl)pyridin-3-yl]methyl}-5-fluoro-2-methylindol-1-yl)-acetic acid;
[5-Fluoro-3-({2-[(4-fluorobenzene)sulfonyl]pyridin-3-yl}methyl)-2-methylindol-1-yl]-acetic acid;
[3-({2-[(4-Chlorobenzene)sulfonyl]pyridin-3-yl}methyl)-5-fluoro-2-methylindol-1-yl]-acetic acid;
2-(3-(4-(Benzylsulfonyl)benzyl)-5-fluoro-2-methyl-indol-1-yl)-acetic acid;
2-(3-(4-(4-Chlorobenzylsulfonyl)benzyl)-5-fluoro-2-methyl-indol-1-yl)-acetic acid;
2-(3-(3-(Benzylsulfonyl)benzyl)-5-fluoro-2-methyl-indol-1-yl)-acetic acid;
2-(5-Fluoro-3-(3-(4-fluorobenzylsulfonyl)benzyl)-2-methyl-indol-1-yl)-acetic acid;
2-(3-(2-(Benzylsulfonyl)benzyl)-5-fluoro-2-methyl-indol-1-yl)-acetic acid;
2-(3-(4-(4-Fluorobenzylsulfonyl)benzyl)-5-fluoro-2-methyl-indol-1-yl)-acetic acid;
2-(3-(2-(Cyclohexylsulfonyl)benzyl)-5-fluoro-2-methyl-indol-1-yl)-acetic acid;
2-(5-Fluoro-2-methyl-3-(2-(piperidin-1-ylsulfonyl)benzyl)-indol-1-yl)-acetic acid;
2-(3-(2-(Cyclopentylsulfonyl)benzyl)-5-fluoro-2-methyl-indol-1-yl)-acetic acid;
2-(5-Fluoro-2-methyl-3-(3-(piperidin-1-ylsulfonyl)benzyl)-indol-1-yl)-acetic acid;
2-(5-Fluoro-2-methyl-3-(2-(pyrrolidin-1-ylsulfonyl)benzyl)-indol-1-yl)-acetic acid;
2-(3-(4-(Cyclohexylsulfonyl)benzyl)-5-fluoro-2-methyl-indol-1-yl)-acetic acid;
2-(3-(4-(Cyclopentylsulfonyl)benzyl)-5-fluoro-2-methyl-indol-1-yl)-acetic acid;
2-(3-(2-(Cyclobutylsulfonyl)benzyl)-5-fluoro-2-methyl-indol-1-yl)-acetic acid;
2-(5-Fluoro-2-methyl-3-(3-(pyrrolidin-1-ylsulfonyl)benzyl)-indol-1-yl)-acetic acid acid;
2-(5-Fluoro-2-methyl-3-(4-(piperidin-1-ylsulfonyl)benzyl)-indol-1-yl)-acetic acid;
[5-Fluoro-2-methyl-3-(2-phenoxybenzyl)-indol-1-yl]-acetic acid;
[5-Fluoro-2-methyl-3-(2-(4-methoxyphenoxy)benzyl)-indol-1-yl]-acetic acid;
[5-Fluoro-2-methyl-3-(2-(4-methylphenoxy)benzyl)-indol-1-yl]-acetic acid;
[5-Fluoro-2-methyl-3-(2-(2,4-dichlorophenoxy)benzyl)-indol-1-yl]-acetic acid;
[5-Fluoro-2-methyl-3-(2-(4-fluorophenoxy)benzyl)-indol-1-yl]-acetic acid;
[5-Fluoro-2-methyl-3-(2-(3,4-difluorophenoxy)benzyl)-indol-1-yl]-acetic acid;
[5-Fluoro-2-methyl-3-(2-(4-cyanophenoxy)benzyl)-indol-1-yl]-acetic acid;
[5-Fluoro-2-methyl-3-(2-(4-chlorophenoxy)benzyl)-indol-1-yl]-acetic acid;
[5-Fluoro-2-methyl-3-(2-(2-cyanophenoxy)benzyl)-indol-1-yl]-acetic acid;
(5-Fluoro-2-methyl-3-{[2-(4-methylphenoxy)pyridin-3-yl]methyl}indol-1-yl)-acetic acid;
{5-Fluoro-3-[(3-methanesulfonylnaphthalen-2-yl)methyl]-2-methylindol-1-yl}-acetic acid;
{5-Fluoro-3-[(1-methanesulfonylnaphthalen-2-yl)methyl]-2-methylindol-1-yl}-acetic acid;
{5-Fluoro-3-[(6-methanesulfonylnaphthalen-2-yl)methyl]-2-methylindol-1-yl}-acetic acid;
[5-Fluoro-2-methyl-3-(quinolin-3-ylmethyl)indol-1-yl]-acetic acid;
[5-Fluoro-2-methyl-3-(quinoxalin-6-ylmethyl)indol-1-yl]-acetic acid;
[5-Fluoro-2-methyl-3-(quinolin-7-ylmethyl)indol-1-yl]-acetic acid;
{5-Fluoro-3-[(6-methanesulfonylquinolin-2-yl)methyl]-2-methylindol-1-yl}-acetic acid;
{5-Fluoro-3-[(4-methanesulfonylquinolin-2-yl)methyl]-2-methylindol-1-yl}-acetic acid;
(5-Fluoro-2-methyl-3-{pyrazolo[1,5-a]pyridin-3-ylmethyl}indol-1-yl)-acetic acid;
(5-Fluoro-3-{imidazo[1,2-a]pyridin-2-ylmethyl}-2-methylindol-1-yl)-acetic acid;
(5-Fluoro-2-methyl-3-{[2-(methylsulfanyl)phenyl]methyl}indol-1-yl)-acetic acid;
(5-Fluoro-2-methyl-3-{[3-(methylsulfanyl)phenyl]methyl}indol-1-yl)-acetic acid;
(5-Fluoro-2-methyl-3-{[4-(ethylsulfanyl)phenyl]methyl}indol-1-yl)-acetic acid(3
{[4-(Ethylsulfanyl)phenyl]methyl}-5-fluoro-2-methylindol-1-yl)-acetic acid;
(5-Fluoro-2-methyl-3-{[4-(n-propylsulfanyl)phenyl]methyl}indol-1-yl)-acetic acid;
(5-Fluoro-2-methyl-3-{[4-(i-propylsulfanyl)phenyl]methyl}indol-1-yl)-acetic acid;
(5-Fluoro-2-methyl-3-{[4-(t-butylsulfanyl)phenyl]methyl}indol-1-yl)-acetic acid;
(5-Fluoro-2-methyl-3-{[4-(pentan-3-ylsulfanyl)phenyl]methyl}indol-1-yl)-acetic acid;
[3-({4-[(Cyclopropylmethyl)sulfanyl]phenyl}methyl)-5-fluoro-2-methylindol-1-yl]-acetic acid;
{3-[(4,4-Dimethyl-2,3-dihydro-1-benzothiopyran-6-yl)methyl]-5-fluoro-2-methylindol-1-yl}-acetic acid;
(3-{[2-(Ethanesulfonyl)phenyl]methyl}-5-fluoro-2-methylindol-1-yl)-acetic acid;
(5-Fluoro-2-methyl-3-{[2-(propane-1-sulfonyl)phenyl]methyl}indol-1-yl)-acetic acid;
(5-Fluoro-2-methyl-3-{[2-(propane-2-sulfonyl)phenyl]methyl}indol-1-yl)-acetic acid;
(3-{[2-(Butane-1-sulfonyl)phenyl]methyl}-5-fluoro-2-methylindol-1-yl)-acetic acid;
(3-{[2-(Butane-2-sulfonyl)phenyl]methyl}-5-fluoro-2-methylindol-1-yl)-acetic acid;
(5-Fluoro-2-methyl-3-{[2-(2-methylpropane-2-sulfonyl)phenyl]methyl}indol-1-yl)-acetic acid;
(5-Fluoro-2-methyl-3-{[2-(pentane-1-sulfonyl)phenyl]methyl}indol-1-yl)-acetic acid;
(3-{[2-(Cyclopropylmethane)sulfonylphenyl]methyl}-5-fluoro-2-methylindol-1-yl)-acetic acid;
(5-Fluoro-2-methyl-3-{[2-(propylsulfamoyl)phenyl]methyl}indol-1-yl)-acetic acid;
(3-{[2-(Butylsulfamoyl)phenyl]methyl}-5-fluoro-2-methylindol-1-yl)-acetic acid;
(5-Fluoro-2-methyl-3-{[3-(propylsulfamoyl)phenyl]methyl}indol-1-yl)-acetic acid;
(3-{[3-(Butylsulfamoyl)phenyl]methyl}-5-fluoro-2-methylindol-1-yl)-acetic acid;
(5-Fluoro-2-methyl-3-{[4-(trifluoromethane)sulfonylphenyl]methyl}indol-1-yl)-acetic acid;
(3-{[4-(Ethanesulfonyl)phenyl]methyl}-5-fluoro-2-methylindol-1-yl)-acetic acid;
(5-Fluoro-2-methyl-3-{[4-(propane-1-sulfonyl)phenyl]methyl}indol-1-yl)-acetic acid;
(5-Fluoro-2-methyl-3-{[4-(propane-2-sulfonyl)phenyl]methyl}indol-1-yl)-acetic acid;
(3-{[4-(Butane-1-sulfonyl)phenyl]methyl}-5-fluoro-2-methylindol-1-yl)-acetic acid;
(5-Fluoro-2-methyl-3-{[4-(2-methylpropane-2-sulfonyl)phenyl]methyl}indol-1-yl)-acetic acid;
(5-Fluoro-2-methyl-3-{[4-(pentane-1-sulfonyl)phenyl]methyl}indol-1-yl)-acetic acid;
(5-Fluoro-2-methyl-3-{[4-(pentan-3-ylsulfonyl)phenyl]methyl}indol-1-yl)-acetic acid;
[3-({4-[(Cyclopropylmethyl)sulfonyl]phenyl}methyl)-5-fluoro-2-methylindol-1-yl]-acetic acid;
(5-Fluoro-2-methyl-3-{[4-(propylsulfamoyl)phenyl]methyl}indol-1-yl)-acetic acid;
(3-{[4-(Butylsulfamoyl)phenyl]methyl}-5-fluoro-2-methylindol-1-yl)-acetic acid;
(5-Fluoro-2-methyl-3-{[4-(trifluoromethoxy)phenyl]methyl}indol-1-yl)-acetic acid;
(5-Fluoro-3-{[4-methanesulfonyl-3-(trifluoromethyl)phenyl]methyl}-2-methylindol-1-yl)-acetic acid;
(5-Fluoro-3-{[4-methanesulfonyl-3-(trifluoromethoxy)phenyl]methyl}-2-methylindol-1-yl)-acetic acid;
{5-Fluoro-3-[(5-methanesulfonylthiophen-2-yl)methyl]-2-methylindol-1-yl}-acetic acid;
{3-[(4,4-dimethyl-1,1-dioxo-2,3-dihydro-1λ⁶-benzothiopyran-6-yl)methyl]-5-fluoro-2-methylindol-1-yl}-acetic acid;
[3-({1-[(4-Chlorobenzene)sulfonyl]pyrrol-2-yl}methyl)-5-fluoro-2-methylindol-1-yl]-acetic acid;
[5-Fluoro-3-({1-[(4-fluorobenzene)sulfonyl]pyrrol-2-yl}methyl)-2-methylindol-1-yl]-acetic acid;
[5-Fluoro-3-({1-[(4-methoxybenzene)sulfonyl]pyrrol-2-yl}methyl)-2-methylindol-1-yl]-acetic acid;
{3-[1-(2,4-Dichloro-benzenesulfonyl)pyrrol-2-ylmethyl]-5-fluoro-2-methyl-indol-1-yl}-acetic acid;
[5-Fluoro-3-({1-[(4-methanesulfonylbenzene)sulfonyl]pyrrol-2-yl}methyl)-2-methylindol-1-yl]acetic acid;
{5-Fluoro-2-methyl-3-[(2-phenylphenyl)methyl]indol-1-yl}-acetic acid;
(3-{[1-(Benzenesulfonyl)indol-2-yl]methyl}-5-fluoro-2-methylindol-1-yl)-acetic acid;
(3-{[2-(4-Chlorophenyl)phenyl]methyl}-5-fluoro-2-methylindol-1-yl)-acetic acid;
(5-Fluoro-2-methyl-3-{[2-(4-methylphenyl)phenyl]methyl}indol-1-yl)-acetic acid;
{5-Fluoro-2-methyl-3-[(3-phenoxyphenyl)methyl]indol-1-yl}-acetic acid;
[5-Fluoro-3-({4-[(4-fluorophenyl)carbonyl]-1-methylpyrrol-2-yl}methyl)-2-methylindol-1-yl]-acetic acid;
{5-Fluoro-2-methyl-3-[(6-{[3-(trifluoromethyl)phenyl]methyl}pyridin-3-yl)methyl]indol-1-yl}-acetic acid;
{-Fluoro-2-methyl-3-[(3-phenoxythiophen-2-yl)methyl]indol-1-yl}-acetic acid;
(3-{[2-(Benzenesulfonyl)-1,3-thiazol-5-yl]methyl}-5-fluoro-2-methylindol-1-yl)-acetic acid;
{3-[(1-Benzylpyrazol-4-yl)methyl]-5-fluoro-2-methylindol-1-yl}-acetic acid;
(3-{[5-(4-Chlorophenoxy)-1-methyl-3-(trifluoromethyl)pyrazol-4-yl]methyl}-5-fluoro-2-methylindol-1-yl)-acetic acid;
[3-({5-[(4-Chlorobenzene)sulfonyl]furan-2-yl}methyl)-5-fluoro-2-methylindol-1-yl]-acetic acid;
[3-({5-[(4-Chlorobenzene)sulfonyl]thiophen-2-yl}methyl)-5-fluoro-2-methylindol-1-yl]-acetic acid;
[3-({3-[(4-Chlorobenzene)sulfonyl]thiophen-2-yl}methyl)-5-fluoro-2-methylindol-1-yl]-acetic acid;
{3-[(2-Benzylphenyl)methyl]-5-fluoro-2-methylindol-1-yl}-acetic acid;
   or the C₁-C₆ alkyl, aryl, (CH₂)ₘOC(═O)C₁-C₆alkyl, ((CH₂)ₘO)ₙCH₂CH₂X, (CH₂)ₘN(R⁷)₂ or CH((CH₂)ₘO(C═O)R⁸)₂ esters of any of the above; wherein
   m is 1 or 2;
   n is 1-4;
   X is OR⁷ or N(R)₂;
   R⁷ is hydrogen or methyl;
   R⁸ is C₁-C₁₈ alkyl.

The compounds of general formula (I) may be prepared according to methods set out in the prior art or methods analogous to those set out in the prior art, in particular WO2005/044260, WO2005/094816 and WO2006/095183, WO2008/012511 and UK patent application Nos 0800874.0, 0801132.2, 0801671.9, 0801131.4, 0801672.7 and 0801674.3.

In a further aspect of the invention, there is provided the use of a CRTH2 antagonist of general formula (I) as defined above in the preparation of an agent for desensitising the immune system of a patient to one or more allergens responsible for an allergic condition.

The invention also provides a method for desensitising the immune system of a patient to one or more allergens responsible for a allergic condition, the method comprising administering to the patient an effective amount of a CRTH2 antagonist of general formula (I) as defined above.

Preferably the CRTH2-related allergic condition is asthma (including allergic asthma), atopic dermatitis, food allergies, acute and chronic utricaria and allergic conjunctivitis perennial allergic rhinitis, seasonal allergic rhinitis, atopic dermatitis, contact hypersensitivity (including contact dermatitis), conjunctivitis, especially allergic conjunctivitis.

Other preferred features are as for the first aspect of the invention.

The compound of general formula (I) may be combined with one or more additional agent which is of use in the treatment of these allergic conditions. Examples of such agents include existing therapies for allergic and other inflammatory diseases including:

Suplatast tosylate and similar compounds;

B₂ adrenoreceptor agonists such as metaproterenol, isoproterenol, isoprenaline, albuterol, salbutamol, formoterol, salmeterol, terbutaline, orciprenaline, bitolterol mesylate, pirbuterol and indacaterol or methylxanthanines such as theophylline and aminophylline, mast cell stabilisers such as sodium cromoglycate or muscarinic receptor antagonists such as ipratropium and tiotropium;
antihistamines, for example histamine H₁ receptor antagonists such as loratadine cetirizine, desloratadine, levocetirizine, fexofenadine, astemizole, azelastine and chlorpheniramine or H₄ receptor antagonists;
α₁ and α₂ adrenoreceptor agonists such as propylhexedrine phenylephrine, phenylpropanolamine, pseudoephedrine, naphazoline hydrochloride, oxymetazoline hydrochloride, tetrahydrozoline hydrochloride, xylometazoline hydrochloride and ethylnorepinephrine hydrochloride;
corticosteroids such as prednisone, prednisolone, flunisolide, triamcinolone acetonide, beclomethasone dipropionate, budesonide, fluticasone propionate, fluticasone furoate, mometasone furoate and ciclesonide;
allergen immunotherapy such as Grazax.

The CRTH2 antagonist of general formula (I) will generally be administered in a pharmaceutical formulation which may be a formulation suitable for oral, rectal, nasal, bronchial (inhaled), topical (including eye drops, buccal and sublingual), vaginal or parenteral (including subcutaneous, intramuscular, intravenous and intradermal) administration and may be prepared by any methods well known in the art of pharmacy.

The route of administration will depend upon the condition to be treated but preferred compositions are formulated for oral, nasal, bronchial or topical administration.

The formulation may be prepared by bringing into association the above defined active agent with a carrier. In general, the formulations are prepared by uniformly and intimately bringing into association the active agent with liquid carriers or finely divided solid carriers or both, and then if necessary shaping the product.

Formulations for oral administration in the present invention may be presented as: discrete units such as capsules, sachets, tablets, which may be chewable tablets, or lozenges, each containing a predetermined amount of the active agent; as a powder or granules; as fine particles for sprinkling over food; as a solution or a suspension of the active agent in an aqueous liquid or a non-aqueous liquid; or as an oil-in-water liquid emulsion or a water in oil liquid emulsion; or as a bolus etc.

For compositions for oral administration (e.g. tablets and capsules), the term “acceptable carrier” includes vehicles such as common excipients e.g. binding agents, for example syrup, acacia, gelatin, sorbitol, tragacanth, polyvinylpyrrolidone (Povidone), methylcellulose, ethylcellulose, sodium carboxymethylcellulose, hydroxypropylmethylcellulose, sucrose and starch; fillers and carriers, for example corn starch, gelatin, lactose, sucrose, microcrystalline cellulose, kaolin, mannitol, dicalcium phosphate, sodium chloride and alginic acid; and lubricants such as magnesium stearate, sodium stearate and other metallic stearates, glycerol stearate stearic acid, silicone fluid, talc waxes, oils and colloidal silica. Flavouring agents such as peppermint, oil of wintergreen, cherry flavouring and the like can also be used. It may be desirable to add a colouring agent to make the dosage form readily identifiable. Tablets may also be coated by methods well known in the art.

A tablet may be made by compression or moulding, optionally with one or more accessory ingredients. Compressed tablets may be prepared by compressing in a suitable machine the active agent in a free flowing form such as a powder or granules, optionally mixed with a binder, lubricant, inert diluent, preservative, surface-active or dispersing agent. Moulded tablets may be made by moulding in a suitable machine a mixture of the powdered compound moistened with an inert liquid diluent. The tablets may optionally be coated or scored and may be formulated so as to provide slow or controlled release of the active agent.

Other formulations suitable for oral administration include lozenges comprising the active agent in a flavoured base, usually sucrose and acacia or tragacanth; pastilles comprising the active agent in an inert base such as gelatin and glycerin, or sucrose and acacia; and mouthwashes comprising the active agent in a suitable liquid carrier. For topical application to the skin, CRTH2 antagonists such as compounds of general formula (I) may be made up into a cream, ointment, jelly, solution or suspension etc. Cream or ointment formulations that may be used for the drug are conventional formulations well known in the art, for example, as described in standard text books of pharmaceutics such as the British Pharmacopoeia.

For topical application to the eye, CRTH2 antagonists such as compounds of general formula (I) may be made up into an eye drop formulation. Suitable eye drop formulations are also well known in the art and are described in standard text books of pharmaceutics such as the British Pharmacopoeia.

CRTH2 antagonists which are intended to reduce or remove the symptoms of conditions such as allergic asthma, allergic conjunctivitis and allergic rhinitis may be administered to the respiratory tract by nasal, bronchial or buccal administration of, for example, aerosols or sprays which can disperse the pharmacological active ingredient in the form of a powder or in the foam of drops of a solution or suspension. Pharmaceutical compositions with powder-dispersing properties usually contain, in addition to the active ingredient, a liquid propellant with a boiling point below room temperature and, if desired, adjuncts, such as liquid or solid non-ionic or anionic surfactants and/or diluents. Pharmaceutical compositions in which the pharmacological active ingredient is in solution contain, in addition to this, a suitable propellant, and furthermore, if necessary, an additional solvent and/or a stabiliser. Instead of the propellant, compressed air can also be used, it being possible for this to be produced as required by means of a suitable compression and expansion device.

Parenteral formulations will generally be sterile.

Typically, the oral dose of the compound will be about 0.01 to 100 mg/kg; so as to achieve a concentration of drug in the plasma at a concentration effective to inhibit PGD₂ at the CRTH2 receptor. The precise amount of a compound of CRTH2 antagonist which is therapeutically effective, and the route by which such compound is best administered, is readily determined by one of ordinary skill in the art by comparing the blood level of the agent to the concentration required to have a therapeutic effect.

In formulations for topical administration, such as eye drops, creams or ointments, the concentration of active compound will typically be about 0.1%-1% w/w such that systemic exposure of the CRTH2 antagonist compound is limited.

Since the mechanism of action appears to be that described above, the prolonged effect on the inflammatory symptoms of allergic conditions will be observed with any CRTH2 antagonist. Also, since the mechanism of action appears to be that described above, the desensitisation effect will be observed with any CRTH2 antagonist. Documents which discuss the use of CRTH2 antagonists for the treatment of Th2-dependent allergic diseases include WO-A-03/066046, WO-A-03/066047, WO-A-03/097042, WO-A-03/097598, WO-A-03/101981, WO-A-03/101961, WO-A-2004/007451, WO-A-2005/019171, WO-A-2005/054232, WO-A-2004/089884, WO-A-2004/089885, WO-A-2005/018529, WO-A-2006/005909, WO2006/021759, WO-A-2007/039736, WO-A-2007/052023, WO-A-2006/075139, WO-A-2007/068894, WO-A-2007138282, WO-A-2008/119917, WO-A-2008/113965, WO-A-2008/074966, WO-A-2008/078069, WO-A-2007/144625, WO-A-2007/028999, WO-A-2007/031747, WO-A-2006/136859, WO-A-2006/111560, WO-A-2005/094816, WO-A-2005/040112, WO-A-2005/040114, WO-A-2004/096777, WO-A-2005/123731, WO-A-2006/125784, WO-A-2007/045867, WO-A-2006/034419, WO-A-2006/036994, WO-A-2007/022501, WO-A-2004/106302, WO-A-2004/032848, WO-A-2005/100321, WO-A-2006/091674, WO-A-2004/058164, WO-A-2005/007094, WO-A-2007/036743, WO-2004/035543, WO-A-2007/062797, WO-A-2007/062773, WO-A-2007/062678, WO-A-2007/062677, WO-A-2005/116001, WO-A-2005/115382, WO-A-2005/115374, WO-A-2006/111560, WO-A-2006/037982, WO-A-2006/056752, WO-A-2007/039741, WO-A-2005/073234, WO-A-2005/105727, WO-A-2006/063763, WO-A-2006/125593 and WO-A-2006/125596. The compounds described in these documents are useful for the treatment of an allergic condition which is induced by the presence of an allergen to which the patient is sensitive, wherein the treatment comprises at least one treatment cycle which comprises:

a. a first period during which the CRTH2 antagonist compound is administered to the patient; and
b. a second period of at least seven days during which the compound of general formula (I) is administered to the patient in a reduced amount;
wherein the allergen is present or suspected to be present throughout the treatment and wherein the CRTH2 antagonist retains at least 50% of its effectiveness during the second period of the treatment cycle. The compounds described in these documents are also useful for desensitising a patient to allergens.

Some particularly suitable compounds are compounds which are similar to the compounds of general formula (I) but in which the moiety —CHR³R⁴ of general formula (I) is replaced by R³, SR³, SOR³ or SO₂R³, where R³ is as defined for general formula (I). An example compound of this type, [5-Fluoro-3-(4-chloro-benzenesulfonyl)-2-methyl-indol-1-yl]-acetic acid, which falls within the scope of WO 2004/007451 was tested in the apoptosis assay of Example 3 (below) and was found counter the rescuing function of PGD₂ with an IC₅₀ of 204±160 nM.

In the context of the present invention, a CRTH2 antagonist is a compound which binds to the CRTH2 receptor with a Ki of less than 1000 nM in the radioligand binding assay described below and which inhibits the dose-dependent increase in intracellular Ca²⁺ mobilisation in CHO/CRTH2 cells treated with PGD₂ with an IC₅₀ of less than 1000 nM. Although any compound with CRTH2 antagonist activity would be suitable for use in desensitising a subject to allergens, the CRTH2 antagonist is suitably a compound of general formula (I).

### EXAMPLES

### Radioligand Binding Assay for Measurement of Binding to CRTH2 Receptor

### Materials

Calcium-3 dye was purchased from Molecular Devices (Wokingham, UK). Mono-poly resolving medium was obtained from Dainippon Pharmaceuticals (Osaka, Japan). Macs anti-CD16 microbeads were from Miltenyi biotec (Bisley, Surrey). ChemoTx plates were purchased from Neuroprobe (Gaithesburg, Md.). Poly-D-lysine coated 96-well plates were obtained from Greiner (Gloucestershire, UK). [³H]PGD₂ was from Amersham Biosciences (Buckinghamshire, UK). [³H]SQ29548 was purchased from Perkin Elmer Life Sciences (Buckinghamshire, UK). All other reagents were obtained from Sigma-Aldrich (Dorset, UK), unless otherwise stated.

### Methods

### Cell Culture

Chinese Hamster Ovary cells were transfected with CRTH2 receptors (CHO/CRTH2) and were maintained in culture in a humidified atmosphere at 37° C. (5% CO₂) in Minimum Essential Medium (MEM) supplemented with 10% foetal bovine serum, 2 mM glutamine, and 1 mg ml⁻¹ active G418. The cells were passaged every 2-3 days. For radioligand binding assay, cells were prepared in triple-layer flasks or in 175 cm² square flasks (for membrane preparation). For calcium mobilisation assay, cells were grown in a 96 well plate 24 h prior to the assay at a density of 80,000 cells per well.

### Preparation of Cell Membranes

Membranes were prepared either from CHO/CRTH2 cells. CHO cells grown to confluency were washed with PBS and detached using a Versene solution (15 ml per flask). When the cells were grown in 175 cm² square flask, they were collected by scrapping in PBS. The cell suspensions were centrifuged (1,700 rpm, 10 min, 4° C.) and resuspended in 15 ml of buffer (1×HBSS, supplemented with 10 mM HEPES, pH 7.3). Cell suspensions were then homogenised using an Ultra Turrax at setting 4-6 for 20 s. The homogenate was centrifuged at 1,700 rpm for 10 min and the supernatant was collected and centrifuged at 20,000 rpm for 1 h at 4° C. The resulting pellet was resuspended in buffer and stored at −80° C. in aliquots of 200-500 μl. The protein concentration was determined by the method of Bradford (1976), using bovine serum albumin as standard. The platelets were washed by centrifugation at 600×g for 10 min and resuspended in ice-cold assay buffer (10 mM Tris-HCl, pH 7.4, 5 mM Glucose, 120 mM NaCl, 10 μM indomethacin) and directly centrifuged at 20,000 rpm for 30 mM at 4° C. The resulting pellet was treated as described above.

### Radioligand Binding Assays

[³H]PGD₂ (160 Ci/mmol) binding experiments were performed on membranes prepared as described above. Assays were performed in a final volume of 100 μl of buffer (1×HBSS/HEPES 10 mM, pH 7.3). Cell membranes (15 μg). Cell membranes 15 mg were preincubated at room temperature with varying concentration of competing ligand for 15 mM. [³H]PGD₂ (mol, final concentration) was then added and the incubation continued for a further one hour at room temperature. The reaction was terminated by the addition of 200 μl ice-cold assay buffer to each well, followed by rapid filtration through Whatman GF/B glass fibre filters using a Unifilter Cell harvester (PerkinElmer Life Sciences) and six washes of 300 μl of ice-cold buffer. The Unifilter plates were dried at room temperature for at least 1 h and the radioactivity retained on the filters was determined on a Beta Trilux counter (PerkinElmer Life Sciences), following addition of 40 μl of Optiphase Hi-Safe 3 (Wallac) liquid scintillation. Non specific binding was defined in the presence of 10 μM unlabelled PGD₂.

This assay is also described in Example 3 of WO 2005/044260. More suitable, compounds which are CRTH2 antagonists have a Ki of less than 100 nM and typically less than 50 nM or even less than 20 nM.

The CRTH2 antagonists useful in the present invention may be selective for the CRTH2 receptor and in this case will not have high affinity for the DP₁ or TP receptors. Binding to the DP₁ receptor may be measured in a radioligand binding assay similar to the one described above for measurement of CRTH2 antagonist activity except that in the cell culture phase, the Chinese Hamster Ovary cells were transfected with DP₁ receptors (CHO/DP), rather than with CRTH2 receptors, and the CHO/DP cells were used to prepare the cell membranes.

Compounds which do not bind to the DP₁ receptor have a Ki of 1 μM or greater in this assay. Indeed, for some compounds the Ki at the DP₁ receptor may be at least 5 μM or even at least 10 μM.

TP receptor radioligand binding may be carried out on membranes prepared from platelets. 15-40 μg of protein were pre-incubated with varying concentrations of competing ligand for 15 min at room temperature in assay buffer (10 mM Tris-HCl, pH 7.4, 5 mM glucose, 120 mM NaCl, 10 μM indomethacin). [³H]SQ29548 (38 Ci/mmol, 10 nM final concentration) was then added and the incubation continued for a further 30 mM at room temperature. The reaction was terminated by the addition of 200 μl ice-cold assay buffer to each well, followed by rapid filtration through Whatman GF/C glass fibre filters using a Unifilter Cell harvester (PerkinElmer Life Sciences) followed with six washes of 300 μl of ice-cold buffer. The radioactivity was determined as described for the CRTH2 and DP₁ receptors.

Compounds useful in the present invention which do not bind to the TP receptor have a Ki of 1 μM or greater in this assay. Indeed, for some compounds the Ki at the TP receptor may be at least 5 μM or even at least 10 μM.

Among the most suitable compounds for use in the present invention are those in which the binding selectivity is greater than 200 fold for CRTH2 receptor, compared to DP₁ TP receptors.

CRTH2 antagonist activity can be measured by the calcium mobilisation assay as follows.

### Calcium Mobilisation Assay For Measurement of CRTH2 Antagonist Activity

Cells were seeded onto poly-D-lysine coated 96-well plates at a density of 80,000 cells per well and incubated at 37° C. overnight to allow the cells to adhere. Cells were washed twice with HBSS and incubated for 1 h at 37° C. in 100 μl HBSS and 100 μl calcium-3-dye (Molecular Devices), supplemented with 4 mM probenecid. Changes in fluorescence were monitored over a 50 s time course with agonist addition at 17 s using a Flexstation (Molecular Devices).

### Effect of CRTH2 Agonists on Calcium Mobilisation in CHO-CRTH2 Cells

PGD₂ caused a dose-dependent increase in intracellular Ca²⁺ mobilisation in CHO/CRTH2 cells, with an EC₅₀=2.4±0.5 nM (n=3).

CRTH2 antagonist compounds inhibit this effect suitably with an IC₅₀ of less than 1 μM, more appropriately less than 500 nM still more suitably less than 100 nM and typically less than 50 nM or even less than 20 nM.

In the Examples, the following test compounds were used.

| | |
|---|---|
| Compound 1 | (5-Fluoro-2-methyl-3-quinolin-2-ylmethyl-indol-1-yl)- |
| | acetic acid |
| Compound 2 | (3-{[2-(Benzenesulfonyl)phenyl]methyl}-5-fluoro-2- |
| | methylindol-1-yl)-acetic acid |
| Compound 3 | {5-Fluoro-3-[(3-methanesulfonylnaphthalen-2-yl)methyl]-2- |
| | methylindol-1-yl}-acetic acid |
| Compound 4 | (3-{[2-(Benzenesulfonyl)pyridin-3-yl]methyl}-5-fluoro-2- |
| | methylindol-1-yl)-acetic acid |
| Compound 5 | [5-Fluoro-3-({2-[(4-fluorobenzene)sulfonyl]pyridin-3- |
| | yl}methyl)-2-methylindol-1-yl]-acetic acid |

### Example 1

### Vienna Challenge Chamber Study

### Study Design

The study was a randomised, double blind, placebo controlled, two way crossover evaluation of Compound 1, given orally for eight days. There was a screening period of one week and a washout period of three weeks between the two treatment periods. There was a follow up one week after the last dose of study drug. The group of patients who received the study drug for the first treatment period and placebo for the second was designated group A, while the group of patients who received placebo for the first treatment period and the study drug for the second treatment period was designated group B.

### Treatment Plan and Methods

The subjects underwent a complete screening assessment to determine a baseline response to allergens. This screening assessment took place one week prior to the start of dosing.
Subjects commenced dosing with Compound 1 or placebo on Day 1 (visit 2 or visit 5) of each treatment period of the study. Adverse events, total nasal symptom score and concomitant medications were noted in the CRF. Subjects took each dose (2 capsules in the morning and 2 capsules in the evening) as soon as they had finished breakfast and their evening meal respectively.

Subjects reported back to the clinic on Day 2 of each treatment period for a 6 hour allergen challenge. The following measurements were obtained:
Total nasal symptom score (TNSS) (obstruction, rhinorrhoea, itch, sneeze) with each symptom scored on a categorical scale from 0 to 3 pre-challenge, every 15 mins from 0 to 6 h post-start of challenge
Eye symptom score (watery eyes, itchy eyes, red eyes) with each symptom scored on a categorical scale from 0 to 3 pre-challenge, every 15 mins from 0 to 6 h post-start of challenge
Other symptoms (cough, itchy throat, itchy ears) with each symptom scored on a categorical scale from 0 to 3 pre-challenge and every 15 mins from 0 to 6 h post-start of challenge

Subjects reported back to the clinic on Day 8 of each treatment period for a 6 hour allergen challenge and the measurements obtained on Day 2 were repeated.

A final follow-up visit was conducted one after the last dose of test article in Treatment Period 2.

### Results

Subjects in Group A received a CRTH2 antagonist for 8 days, followed by a three week washout and then received placebo for 8 days. The subjects showed a reduced response to allergen after dosing with a CRTH2 antagonist. This reduced response to allergen was still present on dosing with placebo. Subjects in Group B received placebo for 8 days, followed by a three week washout and then received a CRTH2 antagonist for 8 days.

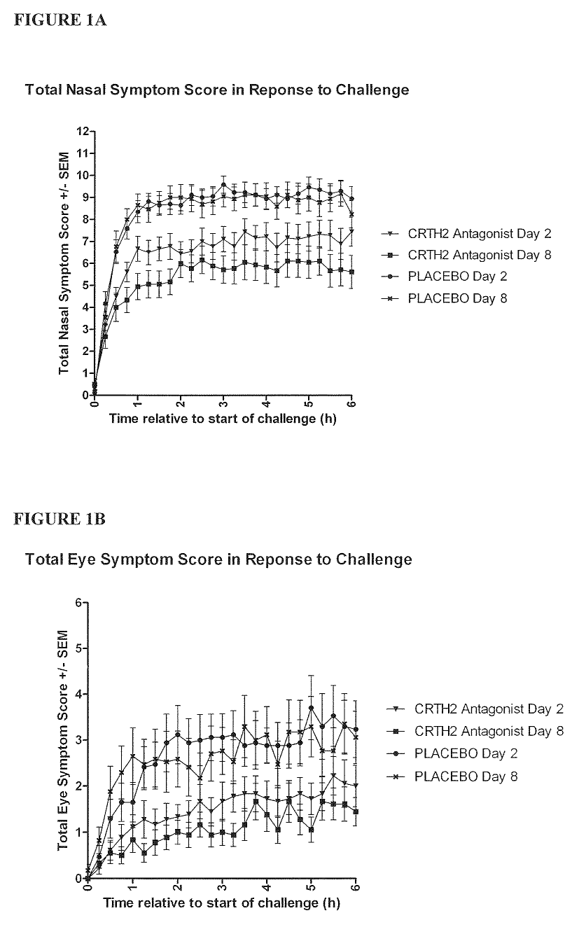

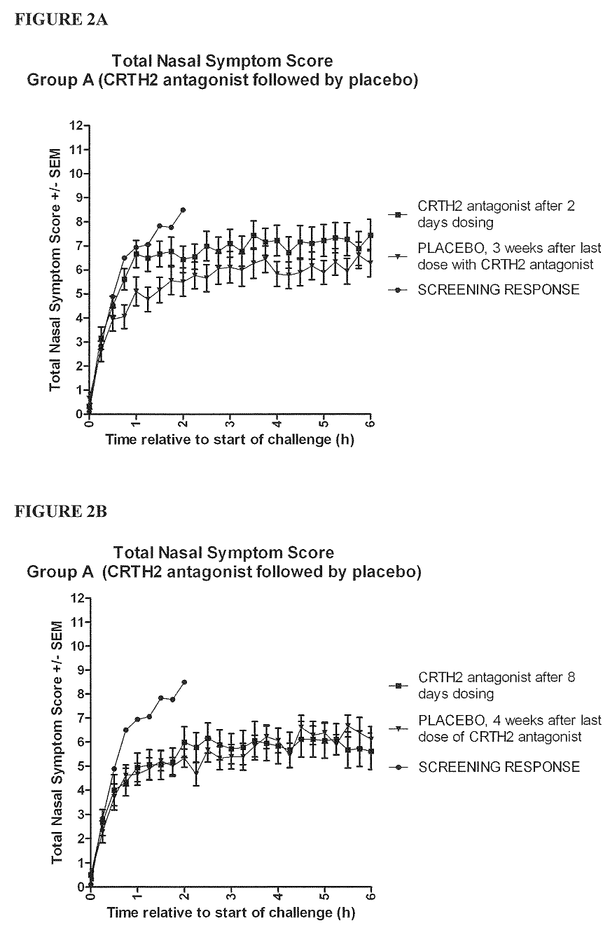

The results of the experiment are shown in FIGS. 1 and 2. When the results for the two groups of patients were combined, subjects show a 23% reduction in total nasal symptom score after 2 days of dosing with a CRTH2 antagonist compared to placebo. This reduction is increased to 37% after 8 days of dosing (FIG. 1A). The total eye symptom score was also reduced in patients treated with the CRTH2 antagonist—subjects show a 45% reduction in total eye symptom score after 2 days of dosing with a CRTH2 antagonist compared to placebo. This reduction is increased to 61% after 8 days of dosing (FIG. 1B).

The figures for the individual patient groups were then obtained. FIGS. 2A and 2B shows that the total nasal symptom scores for patients in Group A on days 2 and 8 respectively of each study period compared with the screening response for these patients obtained at the beginning of the trial. FIGS. 2C and 2D show the equivalent data for Group B.

The subjects mounted the expected response to allergen on placebo and showed a reduction in TNSS in response to allergen after dosing with the CRTH2 antagonist.

The results show that the patients in Group B, who were treated firstly with placebo and then with Compound 1, responded as expected. Thus, as can be seen from FIGS. 2C and 2D, patients treated with placebo showed the same response as the baseline screening response obtained before the beginning of the trial.

However, for patients in Group A, the results were different. As shown in FIGS. 2A and 2B, patients treated with placebo 3 to 4 weeks after the last dose of Compound 1 still had a nasal symptom score which was considerably lower than the baseline value obtained before the beginning of the trial. This indicates that Compound 1 has a long-lasting effect in the patients in Group A and that this effect lasts for more than 4 weeks after the last treatment with Compound 1.

The results demonstrate that in this experiment in the Vienna Challenge Chamber, a reduction of 36% in Total Nasal Symptom Score (0-6 hr) remains 22 days after the last dose of a CRTH2 antagonist and a reduction of 38% in Total Nasal Symptom Score (0-6 hr) remains 28 days after the last dose of a CRTH2 antagonist.

### Example 2

### Measurement of Serum IgE

The effect of the CRTH2 antagonist Compound 1 compared to placebo was studied in mild to moderate asthmatics with an FEV1 of 60-80% of predicted and requiring only short acting inhaled β2-adrenergic agonists for symptomatic control.

Blood was collected for measurement of serum IgE at the beginning of the study and after 4 weeks of dosing with Compound 1 or placebo. Total serum IgE was quantified using fluorescent immunoassay using the Phadia CAP system.

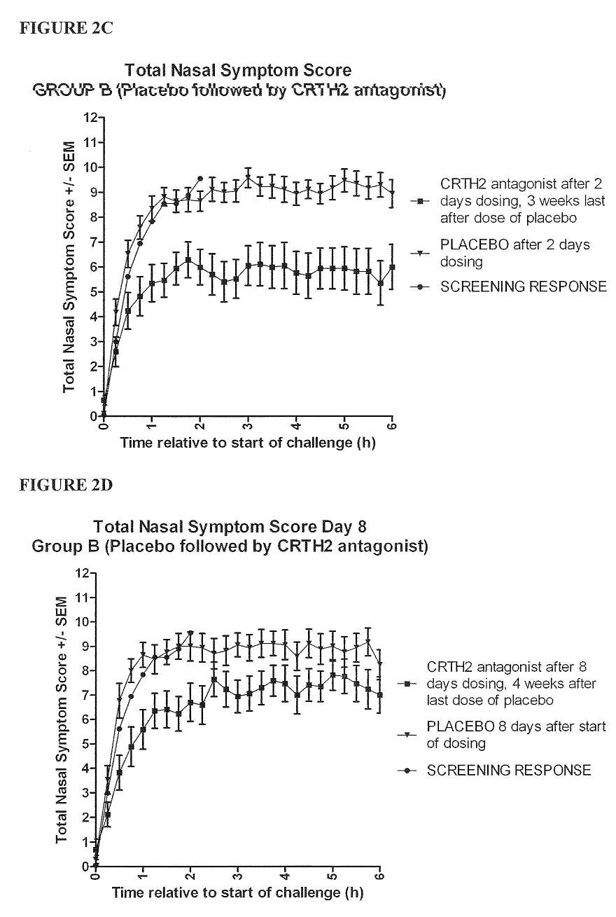

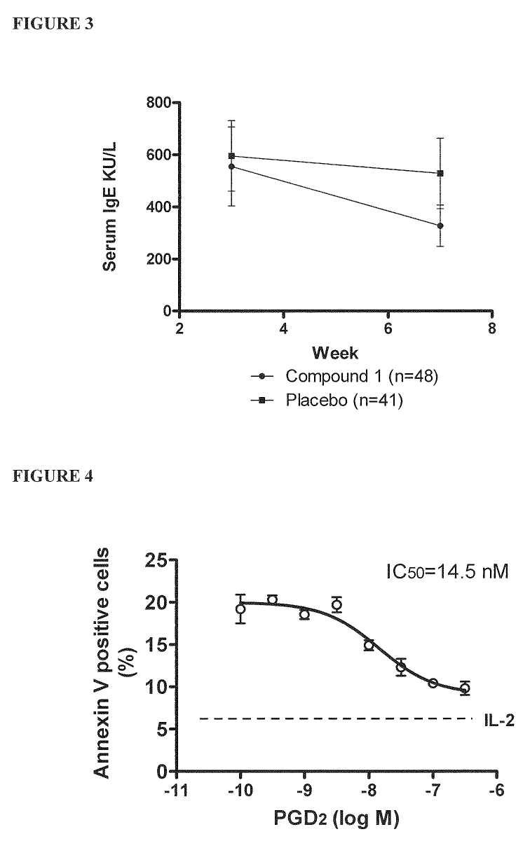

Compound 1 treatment was associated with a significantly greater reduction in IgE than was observed with placebo. The median reduction in IgE at endpoint (−17.0 IU/mL compared with −3.0 IU/mL) was significantly greater with Compound 1 treatment (p=0.033, Wilcoxon test). This equates to a 41% reduction in serum IgE after treatment with Compound 1 for 4 weeks (see FIG. 3).

### Example 3

### Apoptosis Challenge Test

Human Th2 cells were treated with 50 U/ml IL-2 or various concentrations of PGD₂ in the absence of IL-2 for 16 hrs. The cells were stained with Annexin V-PE/PI and then analysed by FACSArray flow cytometer. The results are illustrated in FIG. 3, which shows that PGD₂ has an anti-apoptotic effect on human Th2 cells.

Human Th2 cells were treated in the absence of IL-2 with a medium containing 100 nM PGD₂ and various concentrations of CRTH2 antagonistic compounds (Compounds 1, 2, 3, 4 and 5) for 16 hrs. The cells were stained with Annexin V-PE/PI and then analysed by FACSArray flow cytometer. The IC₅₀ of Compounds 1 to 5 to the rescuing function of PGD₂ were calculated and found to be less than 100 nM in all cases.

The results of this experiment show that while PGD2 has an anti-aptotic effect on Th2 cells, the CRTH2 antagonists have a pro-apoptotic effect.

Having now fully described this invention, it will be understood by those of ordinary skill in the art that the same can be performed within a wide and equivalent range of conditions, formulations and other parameters without affecting the scope of the invention or any embodiment thereof. All patents, patent applications, and publications cited herein are fully incorporated by reference in their entirety.

1. A method of preventing, treating, or ameliorating an allergic condition in an individual, comprising administering to the individual a therapeutically effective amount of a compound of general formula (I): 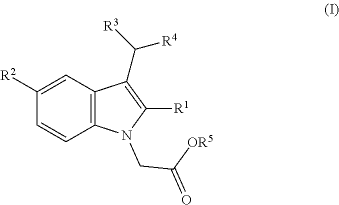
wherein
R¹ is C₁-C₆ alkyl;
R² is halogen;
R³ is aryl or heteroaryl optionally substituted with one or more substituents selected from halo, OH, CN, R⁶, COR⁶, CH₂R⁶, OR⁶, SR⁶, SO₂R⁶, or SO₂YR⁶;
R⁶ is C₁-C₆ alkyl, C₃-C₈ cycloalkyl, heterocyclyl, aryl, or heteroaryl, any of which may optionally be substituted with one or more substituents selected from halo, OH, CN, NO₂, C₁-C₆ alkyl, or O(C₁-C₆ alkyl); and
Y is NH or a straight or branched C₁-C₄ alkylene chain;
R⁴ is H or C₁-C₄ alkyl; and
R⁵ is hydrogen, C₁-C₆ alkyl, aryl, (CH₂)ₘOC(═O)C₁-C₆alkyl, ((CH₂)ₘO)ₙCH₂CH₂X, (CH₂)ₘN(R⁷)₂, or CH((CH₂)ₘO(C═O)R⁸)₂;
m is 1 or 2;
n is 1-4;
X is OR⁷ or N(R⁷)₂;
R⁷ is hydrogen or methyl;
R⁸ is C₁-C₁₈ alkyl;
or a pharmaceutically acceptable salt, hydrate, solvate, or complex thereof;
wherein the therapeutically effective amount is administered for at least one treatment cycle comprising:
(a) a first period during which the compound of general formula (I) is administered to the patient; and
(b) a second period of at least seven days during which the compound of general formula (I) is administered to the patient in a reduced amount;
wherein the compound of general formula (I) retains at least 50% effectiveness during the second period of the treatment cycle.

2. The method of claim 1, wherein R⁵ is hydrogen.

3. The method of claim 1, wherein R⁵ is C₁-C₆ alkyl, aryl, (CH₂)ₘOC(═O)C₁-C₆alkyl, ((CH₂)ₘO)ₙCH₂CH₂X, (CH₂)ₘN(R⁷)₂, or CH((CH₂)ₘO(C═O)R⁸)₂.

4. The method of claim 1, wherein
R¹ is C₁-C₄ alkyl;
R² is fluoro;
R³ is optionally substituted and is quinoline, quinoxaline, isoquinoline, thiazole, phenyl, naphthalene, thiophene, pyrrole, or pyridine; and
R⁴ is H or methyl.

5. The method of claim 4, wherein R⁴ is H.

6. The method of claim 1, wherein R³ is optionally substituted and is quinoline, isoquinoline, phenyl, naphthalene, thiophene, pyrrole, or pyridine.

7. The method of claim 6, wherein R³ is quinoline or isoquinoline, wherein the quinoline or isoquinoline is unsubstituted or substituted with one or more halo substituents.

8. The method of claim 6, wherein R³ is optionally substituted with one or more substituents and is phenyl, naphthalene, thiophene, pyrrole, or pyridine, wherein the one or more substituents are OR⁶, SO₂R⁶ or SO₂YR⁶.

9. The method of claim 1, wherein R⁶ is optionally substituted and is C₁-C₆ alkyl, a 4- to 6-membered cycloalkyl group, a 5- or 6-membered heterocyclyl group, or phenyl.

10. The method of claim 6, wherein R³ is a 3-pyridyl moiety.

11. The method of claim 1, wherein R³ is substituted with SO₂YR⁶, wherein Y is a CH₂ moiety.

12. The method of claim 1, wherein R³ is substituted with SO₂R⁶ or SO₂YR⁶, wherein the R⁶ group is unsubstituted or substituted with one or more substituents selected from methyl and halo.

13. The method of claim 1, wherein R³ is substituted with OR⁶, wherein the R⁶ group is unsubstituted or substituted with one or more substituents selected from the group consisting of halo, cyano, C₁-C₄ alkyl, and O(C₁-C₄ alkyl).

14. The method of claim 1, wherein the compound of general formula (I) is:
{3-[1-(4-Chloro-phenyl)-ethyl]-5-fluoro-2-methyl-indol-1-yl}-acetic acid;
{5-Fluoro-2-methyl-3-[1-(4-trifluoromethyl-phenyl)-ethyl]-indol-1-yl}-acetic acid;
{3-[1-(4-tert-Butyl-phenyl)-ethyl]-5-fluoro-2-methyl-indol-1-yl}-acetic acid;
{5-Fluoro-3-[1-(4-methanesulfonyl-phenyl)-ethyl]-2-methyl-indol-1-yl}-acetic acid;
[5-Fluoro-2-methyl-3-(1-naphthalen-2-yl-ethyl)-indol-1-yl]-acetic acid;
(5-Fluoro-2-methyl-3-quinolin-2-ylmethyl-indol-1-yl)-acetic acid;
(5-Fluoro-2-methyl-3-naphthalen-2-ylmethyl-indol-1-yl)-acetic acid;
[5-Fluoro-3-(8-hydroxyquinolin-2-ylmethyl)-2-methyl-indol-1-yl]-acetic acid;
[5-Fluoro-2-methyl-3-(quinoxalin-2-ylmethyl)indol-1-yl]-acetic acid;
[5-Fluoro-3-(4-methoxy-benzyl)-2-methyl-indol-1-yl]-acetic acid;
[5-Fluoro-2-methyl-3-(1,3-thiazol-2-ylmethyl)indol-1-yl]-acetic acid;
[3-(4-Chloro-benzyl)-5-fluoro-2-methyl-indol-1-yl]-acetic acid;
[5-Fluoro-2-methyl-3-(4-trifluoromethyl-benzyl)-indol-1-yl]-acetic acid;
[5-Fluoro-2-methyl-3-(4-tert-butyl-benzyl)-indol-1-yl]-acetic acid;
{5-Fluoro-2-methyl-3-[(4-phenylphenyl)methyl]indol-1-yl}-acetic acid;
[5-Fluoro-3-(4-methanesulfonyl-benzyl)-2-methyl-indol-1-yl]-acetic acid;
{5-Fluoro-3-[(6-fluoroquinolin-2-yl)methyl]-2-methylindol-1-yl}-acetic acid;
(2-Methyl-3-quinolin-2-ylmethyl-indol-1-yl)-acetic acid;
(5-Chloro-2-methyl-3-quinolin-2-ylmethyl-indol-1-yl)-acetic acid;
(3-{[1-(Benzenesulfonyl)pyrrol-2-yl]methyl}-5-fluoro-2-methylindol-1-yl)-acetic acid;
[5-Fluoro-2-methyl-3-({1-[(4-methylbenzene)sulfonyl]pyrrol-2-yl}methyl)indol-1-yl]-acetic acid;
[3-({1-[(2,4-Difluorobenzene)sulfonyl]pyrrol-2-yl}methyl)-5-fluoro-2-methylindol-1-yl]-acetic acid;
(3-{[2-(Benzenesulfonyl)phenyl]methyl}-5-fluoro-2-methylindol-1-yl)-acetic acid;
[3-({2-[(4-Chlorobenzene)sulfonyl]phenyl}methyl)-5-fluoro-2-methylindol-1-yl]-acetic acid;
[5-Fluoro-3-({2-[(4-fluorobenzene)sulfonyl]phenyl}methyl)-2-methylindol-1-yl]-acetic acid;
(3-{[2-(Benzenesulfonyl)pyridin-3-yl]methyl}-5-fluoro-2-methylindol-1-yl)-acetic acid;
[5-Fluoro-3-({2-[(4-fluorobenzene)sulfonyl]pyridin-3-yl}methyl)-2-methylindol-1-yl]-acetic acid;
[3-({2-[(4-Chlorobenzene)sulfonyl]pyridin-3-yl}methyl)-5-fluoro-2-methylindol-1-yl]-acetic acid;
2-(3-(4-(Benzylsulfonyl)benzyl)-5-fluoro-2-methyl-indol-1-yl)-acetic acid;
2-(3-(4-(4-Chlorobenzylsulfonyl)benzyl)-5-fluoro-2-methyl-indol-1-yl)-acetic acid;
2-(3-(3-(Benzylsulfonyl)benzyl)-5-fluoro-2-methyl-indol-1-yl)-acetic acid;
2-(5-Fluoro-3-(3-(4-fluorobenzylsulfonyl)benzyl)-2-methyl-indol-1-yl)-acetic acid;
2-(3-(2-(Benzylsulfonyl)benzyl)-5-fluoro-2-methyl-indol-1-yl)-acetic acid;
2-(3-(4-(4-Fluorobenzylsulfonyl)benzyl)-5-fluoro-2-methyl-indol-1-yl)-acetic acid;
2-(3-(2-(Cyclohexylsulfonyl)benzyl)-5-fluoro-2-methyl-indol-1-yl)-acetic acid;
2-(5-Fluoro-2-methyl-3-(2-(piperidin-1-ylsulfonyl)benzyl)-indol-1-yl)-acetic acid;
2-(3-(2-(Cyclopentylsulfonyl)benzyl)-5-fluoro-2-methyl-indol-1-yl)-acetic acid;
2-(5-Fluoro-2-methyl-3-(3-(piperidin-1-ylsulfonyl)benzyl)-indol-1-yl)-acetic acid;
2-(5-Fluoro-2-methyl-3-(2-(pyrrolidin-1-ylsulfonyl)benzyl)-indol-1-yl)-acetic acid;
2-(3-(4-(Cyclohexylsulfonyl)benzyl)-5-fluoro-2-methyl-indol-1-yl)-acetic acid;
2-(3-(4-(Cyclopentylsulfonyl)benzyl)-5-fluoro-2-methyl-indol-1-yl)-acetic acid;
2-(3-(2-(Cyclobutylsulfonyl)benzyl)-5-fluoro-2-methyl-indol-1-yl)-acetic acid;
2-(5-Fluoro-2-methyl-3-(3-(pyrrolidin-1-ylsulfonyl)benzyl)-indol-1-yl)-acetic acid;
2-(5-Fluoro-2-methyl-3-(4-(piperidin-1-ylsulfonyl)benzyl)-indol-1-yl)-acetic acid;
[5-Fluoro-2-methyl-3-(2-phenoxybenzyl)-indol-1-yl]-acetic acid;
[5-Fluoro-2-methyl-3-(2-(4-methoxyphenoxy)benzyl)-indol-1-yl]-acetic acid;
[5-Fluoro-2-methyl-3-(2-(4-methylphenoxy)benzyl)-indol-1-yl]-acetic acid;
[5-Fluoro-2-methyl-3-(2-(2,4-dichlorophenoxy)benzyl)-indol-1-yl]-acetic acid;
[5-Fluoro-2-methyl-3-(2-(4-fluorophenoxy)benzyl)-indol-1-yl]-acetic acid;
[5-Fluoro-2-methyl-3-(2-(3,4-difluorophenoxy)benzyl)-indol-1-yl]-acetic acid;
[5-Fluoro-2-methyl-3-(2-(4-cyanophenoxy)benzyl)-indol-1-yl]-acetic acid;
[5-Fluoro-2-methyl-3-(2-(4-chlorophenoxy)benzyl)-indol-1-yl]-acetic acid;
[5-Fluoro-2-methyl-3-(2-(2-cyanophenoxy)benzyl)-indol-1-yl]-acetic acid;
(5-Fluoro-2-methyl-3-{[2-(4-methylphenoxy)pyridin-3-yl]methyl}indol-1-yl)-acetic acid;
{5-Fluoro-3-[(3-methanesulfonylnaphthalen-2-yl)methyl]-2-methylindol-1-yl}-acetic acid;
{5-Fluoro-3-[(1-methanesulfonylnaphthalen-2-yl)methyl]-2-methylindol-1-yl}-acetic acid;
{5-Fluoro-3-[(6-methanesulfonylnaphthalen-2-yl)methyl]-2-methylindol-1-yl}-acetic acid;
[5-Fluoro-2-methyl-3-(quinolin-3-ylmethyl)indol-1-yl]-acetic acid;
[5-Fluoro-2-methyl-3-(quinoxalin-6-ylmethyl)indol-1-yl]-acetic acid;
[5-Fluoro-2-methyl-3-(quinolin-7-ylmethyl)indol-1-yl]-acetic acid;
{5-Fluoro-3-[(6-methanesulfonylquinolin-2-yl)methyl]-2-methylindol-1-yl}-acetic acid;
{5-Fluoro-3-[(4-methanesulfonylquinolin-2-yl)methyl]-2-methylindol-1-yl}-acetic acid;
(5-Fluoro-2-methyl-3-{pyrazolo[1,5-a]pyridin-3-ylmethyl}indol-1-yl)-acetic acid;
(5-Fluoro-3-{imidazo[1,2-a]pyridin-2-ylmethyl}-2-methylindol-1-yl)-acetic acid;
(5-Fluoro-2-methyl-3-{[2-(methylsulfanyl)phenyl]methyl}indol-1-yl)-acetic acid;
(5-Fluoro-2-methyl-3-{[3-(methylsulfanyl)phenyl]methyl}indol-1-yl)-acetic acid;
(5-Fluoro-2-methyl-3-{[4-(ethylsulfanyl)phenyl]methyl}indol-1-yl)-acetic acid;
(3-{[4-(Ethylsulfanyl)phenyl]methyl}-5-fluoro-2-methylindol-1-yl)-acetic acid;
(5-Fluoro-2-methyl-3-{[4-(n-propyl sulfanyl)phenyl]methyl}indol-1-yl)-acetic acid;
(5-Fluoro-2-methyl-3-{[4-(i-propylsulfanyl)phenyl]methyl}indol-1-yl)-acetic acid;
(5-Fluoro-2-methyl-3-{[4-(t-butylsulfanyl)phenyl]methyl}indol-1-yl)-acetic acid;
(5-Fluoro-2-methyl-3-{[4-(pentan-3-ylsulfanyl)phenyl]methyl}indol-1-yl)-acetic acid;
[3-({4-[(Cyclopropylmethyl)sulfanyl]phenyl}methyl)-5-fluoro-2-methylindol-1-yl]-acetic acid;
{3-[(4,4-Dimethyl-2,3-dihydro-1-benzothiopyran-6-yl)methyl]-5-fluoro-2-methylindol-1-yl}-acetic acid;
(3-{[2-(Ethanesulfonyl)phenyl]methyl}-5-fluoro-2-methylindol-1-yl)-acetic acid;
(5-Fluoro-2-methyl-3-{[2-(propane-1-sulfonyl)phenyl]methyl}indol-1-yl)-acetic acid;
(5-Fluoro-2-methyl-3-{[2-(propane-2-sulfonyl)phenyl]methyl}indol-1-yl)-acetic acid;
(3-{[2-(Butane-1-sulfonyl)phenyl]methyl}-5-fluoro-2-methylindol-1-yl)-acetic acid;
(3-{[2-(Butane-2-sulfonyl)phenyl]methyl}-5-fluoro-2-methylindol-1-yl)-acetic acid;
(5-Fluoro-2-methyl-3-{[2-(2-methylpropane-2-sulfonyl)phenyl]methyl}indol-1-yl)-acetic acid;
(5-Fluoro-2-methyl-3-{[2-(pentane-1-sulfonyl)phenyl]methyl}indol-1-yl)-acetic acid;
(3-{[2-(Cyclopropylmethane)sulfonylphenyl]methyl}-5-fluoro-2-methylindol-1-yl)-acetic acid;
(5-Fluoro-2-methyl-3-{[2-(propylsulfamoyl)phenyl]methyl}indol-1-yl)-acetic acid;
(3-{[2-(Butylsulfamoyl)phenyl]methyl}-5-fluoro-2-methylindol-1-yl)-acetic acid;
(5-Fluoro-2-methyl-3-{[3-(propylsulfamoyl)phenyl]methyl}indol-1-yl)-acetic acid;
(3-{[3-(Butylsulfamoyl)phenyl]methyl}-5-fluoro-2-methylindol-1-yl)-acetic acid;
(5-Fluoro-2-methyl-3-{[4-(trifluoromethane)sulfonylphenyl]methyl}indol-1-yl)-acetic acid;
(3-{[4-(Ethanesulfonyl)phenyl]methyl}-5-fluoro-2-methylindol-1-yl)-acetic acid;
(5-Fluoro-2-methyl-3-{[4-(propane-1-sulfonyl)phenyl]methyl}indol-1-yl)-acetic acid;
(5-Fluoro-2-methyl-3-{[4-(propane-2-sulfonyl)phenyl]methyl}indol-1-yl)-acetic acid;
(3-{[4-(Butane-1-sulfonyl)phenyl]methyl}-5-fluoro-2-methylindol-1-yl)-acetic acid;
(5-Fluoro-2-methyl-3-{[4-(2-methylpropane-2-sulfonyl)phenyl]methyl}indol-1-yl)-acetic acid;
(5-Fluoro-2-methyl-3-{[4-(pentane-1-sulfonyl)phenyl]methyl}indol-1-yl)-acetic acid;
(5-Fluoro-2-methyl-3-{[4-(pentan-3-ylsulfonyl)phenyl]methyl}indol-1-yl)-acetic acid;
[3-({4-[(Cyclopropylmethyl)sulfonyl]phenyl}methyl)-5-fluoro-2-methylindol-1-yl]-acetic acid;
(5-Fluoro-2-methyl-3-{[4-(propylsulfamoyl)phenyl]methyl}indol-1-yl)-acetic acid;
(3-{[4-(Butylsulfamoyl)phenyl]methyl}-5-fluoro-2-methylindol-1-yl)-acetic acid;
(5-Fluoro-2-methyl-3-{[4-(trifluoromethoxy)phenyl]methyl}indol-1-yl)-acetic acid;
(5-Fluoro-3-{[4-methanesulfonyl-3-(trifluoromethyl)phenyl]methyl}-2-methylindol-1-yl)-acetic acid;
(5-Fluoro-3-{[4-methanesulfonyl-3-(trifluoromethoxy)phenyl]methyl}-2-methylindol-1-yl)-acetic acid;
{5-Fluoro-3-[(5-methanesulfonylthiophen-2-yl)methyl]-2-methylindol-1-yl}-acetic acid;
{3-[(4,4-dimethyl-1,1-dioxo-2,3-dihydro-1λ⁶-benzothiopyran-6-yl)methyl]-5-fluoro-2-methylindol-1-yl}-acetic acid;
[3-({1-[(4-Chlorobenzene)sulfonyl]pyrrol-2-yl}methyl)-5-fluoro-2-methylindol-1-yl]-acetic acid;
[5-Fluoro-3-({1-[(4-fluorobenzene)sulfonyl]pyrrol-2-yl}methyl)-2-methylindol-1-yl]-acetic acid;
[5-Fluoro-3-({1-[(4-methoxybenzene)sulfonyl]pyrrol-2-yl}methyl)-2-methylindol-1-yl]-acetic acid;
{3-[1-(2,4-Dichloro-benzene sulfonyl)pyrrol-2-ylmethyl]-5-fluoro-2-methyl-indol-1-yl}-acetic acid;
[5-Fluoro-3-({1-[(4-methanesulfonylbenzene)sulfonyl]pyrrol-2-yl}methyl)-2-methylindol-1-yl]-acetic acid;
{5-Fluoro-2-methyl-3-[(2-phenyphenyl)methyl]indol-1-yl}-acetic acid;
(3-{[1-(Benzene sulfonyl)indol-2-yl]methyl}-5-fluoro-2-methyl indol-1-yl)-acetic acid;
(3-{[2-(4-Chlorophenyl)phenyl]methyl}-5-fluoro-2-methylindol-1-yl)-acetic acid;
(5-Fluoro-2-methyl-3-{[2-(4-methylphenyl)phenyl]methyl}indol-1-yl)-acetic acid;
{5-Fluoro-2-methyl-3-[(3-phenoxyphenyl)methyl]indol-1-yl}-acetic acid;
[5-Fluoro-3-({4-[(4-fluorophenyl)carbonyl]-1-methylpyrrol-2-yl}methyl)-2-methylindol-1-yl]-acetic acid;
{5-Fluoro-2-methyl-3-[(6-{[3-(trifluoromethyl)phenyl]methyl}pyridin-3-yl)methyl]indol-1-yl}-acetic acid;
{5-Fluoro-2-methyl-3-[(3-phenoxythiophen-2-yl)methyl]indol-1-yl}-acetic acid;
(3-{[2-(Benzene sulfonyl)-1,3-thiazol-5-yl]methyl}-5-fluoro-2-methylindol-1-yl)-acetic acid;
{3-[(1-Benzylpyrazol-4-yl)methyl]-5-fluoro-2-methylindol-1-yl}-acetic acid;
(3-{[5-(4-Chlorophenoxy)-1-methyl-3-(trifluoromethyl)pyrazol-4-yl]methyl}-5-fluoro-2-methylindol-1-yl)-acetic acid;
[3-({5-[(4-Chlorobenzene)sulfonyl]furan-2-yl}methyl)-5-fluoro-2-methyl indol-1-yl]-acetic acid;
[3-({5-[(4-Chlorobenzene)sulfonyl]thiophen-2-yl}methyl)-5-fluoro-2-methylindol-1-yl]-acetic acid;
[3-({3-[(4-Chlorobenzene)sulfonyl]thiophen-2-yl}methyl)-5-fluoro-2-methyl indol-1-yl]-acetic acid;
{3-[(2-Benzylphenyl)methyl]-5-fluoro-2-methylindol-1-yl}-acetic acid;
or the C₁-C₆ alkyl, aryl, (CH₂)ₘOC(═O)C₁-C₆alkyl, ((CH₂)ₘO)ₙCH₂CH₂X, (CH₂)ₘN(R⁷)₂, or CH((CH₂)ₘO(C═O)R⁸)₂ esters of any of the above; wherein
m is 1 or 2;
n is 1-4;
X is OR⁷ or N(R⁷)₂;
R⁷ is hydrogen or methyl;
R⁸ is C₁-C₁₈ alkyl.

15. The method of claim 1, wherein the compound of general formula (I) is administered in combination with one or more additional agent which is of use in the treatment of allergic conditions.

16. The method of claim 15, wherein the additional agent is selected from the group consisting of:
Suplatast tosylate and similar compounds;
B₂ adrenoreceptor agonists such as metaproterenol, isoproterenol, isoprenaline, albuterol, salbutamol, formoterol, salmeterol, terbutaline, orciprenaline, bitolterol mesylate, pirbuterol and indacaterol or methylxanthanines such as theophylline and aminophylline, mast cell stabilisers such as sodium cromoglycate or muscarinic receptor antagonists such as ipratropium and tiotropium;
antihistamines, for example histamine H₁ receptor antagonists such as loratadine cetirizine, desloratadine, levocetirizine, fexofenadine, astemizole, azelastine and chlorpheniramine or H₄ receptor antagonists;
α₁ and α₂ adrenoreceptor agonists such as propylhexedrine phenylephrine, phenylpropanolamine, pseudoephedrine, naphazoline hydrochloride, oxymetazoline hydrochloride, tetrahydrozoline hydrochloride, xylometazoline hydrochloride and ethylnorepinephrine hydrochloride;
corticosteroids such as prednisone, prednisolone, flunisolide, triamcinolone acetonide, beclomethasone dipropionate, budesonide, fluticasone propionate, fluticasone furoate, mometasone furoate and ciclesonide; and
allergen immunotherapy such as Grazax.

17. The method of claim 1, wherein the compound of general formula (I) is administered via the oral, nasal, bronchial, or topical route.

18. The method of claim 1, wherein the individual experiences a reduction in symptoms of at least 50% during the first period of the treatment cycle.

19. The method of claim 1, wherein the compound of general formula (I) retains at least 80% effectiveness during the second period of the treatment cycle.

20. The method of claim 1, wherein the compound of general formula (I) is not administered during the second period of the treatment cycle.

21. The method of claim 1, wherein the second period of the treatment cycle is at least 28 days.

22. The method of claim 1, wherein the first period of the treatment cycle is from two to eight days.

23. The method of claim 1, wherein the first period further comprises administering the compound of general formula (I) once daily.

24. The method of claim 1, further comprising one or more additional treatment cycles.

25. The method of claim 1, wherein the allergic condition is selected from the group consisting of asthma, allergic asthma, food allergies, acute and chronic urticaria, perennial allergic rhinitis, seasonal allergic rhinitis, atopic dermatitis, contact hypersensitivity, contact dermatitis, conjunctivitis, and allergic conjunctivitis.

26. A method for the prolonged reduction of the severity of the symptoms produced by an allergen in a patient, comprising:
(a) administering a therapeutic amount of a compound of general formula (I): 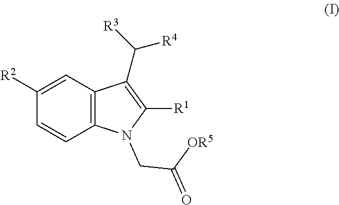
wherein
R¹ is C₁-C₆ alkyl;
R² is halogen;
R³ is aryl or heteroaryl optionally substituted with one or more substituents selected from halo, OH, CN, R⁶, COR⁶, CH₂R⁶, OR⁶, SR⁶, SO₂R⁶, or SO₂YR⁶;
R⁶ is C₁-C₆ alkyl, C₃-C₈ cycloalkyl, heterocyclyl, aryl, or heteroaryl, any of which may optionally be substituted with one or more substituents selected from halo, OH, CN, NO₂, C₁-C₆ alkyl, or O(C₁-C₆ alkyl); and
Y is NH or a straight or branched C₁-C₄ alkylene chain;
R⁴ is H or C₁-C₄ alkyl; and
R⁵ is hydrogen, C₁-C₆ alkyl, aryl, (CH₂)ₘOC(═O)C₁-C₆alkyl, ((CH₂)ₘO)ₙCH₂CH₂X, (CH₂)ₘN(R⁷)₂, or CH((CH₂)ₘO(C═O)R⁸)₂;
m is 1 or 2;
n is 1-4;
X is OR⁷ or N(R⁷)₂;
R⁷ is hydrogen or methyl;
R⁸ is C₁-C₁₈ alkyl;
or a pharmaceutically acceptable salt, hydrate, solvate, or complex thereof; and
(b) ceasing administration of said compound of general formula (I);
wherein the symptoms of the allergic condition continue to be reduced after said compound of general formula (I) is no longer in the body of the patient.

27. The method of claim 26, wherein R⁵ is hydrogen.

28. The method of claim 26, wherein R⁵ is C₁-C₆ alkyl, aryl, (CH₂)ₘOC(═O)C₁-C₆alkyl, ((CH₂)ₘO)ₙCH₂CH₂X, (CH₂)ₘN(R⁷)₂, or CH((CH₂)ₘO(C═O)R⁸)₂.

29. The method of claim 26, wherein
R¹ is C₁-C₄ alkyl;
R² is fluoro;
R³ is optionally substituted and is quinoline, quinoxaline, isoquinoline, thiazole, phenyl, naphthalene, thiophene, pyrrole, or pyridine; and
R⁴ is H or methyl.

30. The method of claim 29, wherein R⁴ is H.

31. The method of claim 26, wherein R³ is optionally substituted and is quinoline, isoquinoline, phenyl, naphthalene, thiophene, pyrrole, or pyridine.

32. The method of claim 31, wherein R³ is quinoline or isoquinoline, wherein the quinoline or isoquinoline is unsubstituted or substituted with one or more halo substituents.

33. The method of claim 31, wherein R³ is optionally substituted with one or more substituents and is phenyl, naphthalene, thiophene, pyrrole, or pyridine, wherein the one or more substituents are OR⁶, SO₂R⁶, or SO₂YR⁶.

34. The method of claim 26, wherein R⁶ is optionally substituted and is C₁-C₆ alkyl, a 4- to 6-membered cycloalkyl group, a 5- or 6-membered heterocyclyl group, or phenyl.

35. The method of claim 31, wherein R³ is a 3-pyridyl moiety.

36. The method of claim 26, wherein R³ is substituted with SO₂YR⁶, wherein Y is a CH₂ moiety.

37. The method of claim 26, wherein R³ is substituted with SO₂R⁶ or SO₂YR⁶, wherein the R⁶ group is unsubstituted or substituted with one or more substituents selected from methyl and halo.

38. The method of claim 26, wherein R³ is substituted with OR⁶, wherein the R⁶ group is unsubstituted or substituted with one or more substituents chosen from the group selected from halo, cyano, C₁-C₄ alkyl, and O(C₁-C₄ alkyl).

39. The method of claim 26, wherein the compound of general formula (I) is:
{3-[1-(4-Chloro-phenyl)-ethyl]-5-fluoro-2-methyl-indol-1-yl}-acetic acid;
{5-Fluoro-2-methyl-3-[1-(4-trifluoromethyl-phenyl)-ethyl]-indol-1-yl}-acetic acid;
{3-[1-(4-tert-Butyl-phenyl)-ethyl]-5-fluoro-2-methyl-indol-1-yl}-acetic acid;
{5-Fluoro-3-[1-(4-methanesulfonyl-phenyl)-ethyl]-2-methyl-indol-1-yl}-acetic acid;
[5-Fluoro-2-methyl-3-(1-naphthalen-2-yl-ethyl)-indol-1-yl]-acetic acid;
(5-Fluoro-2-methyl-3-quinolin-2-ylmethyl-indol-1-yl)-acetic acid;
(5-Fluoro-2-methyl-3-naphthalen-2-ylmethyl-indol-1-yl)-acetic acid;
[5-Fluoro-3-(8-hydroxyquinolin-2-ylmethyl)-2-methyl-indol-1-yl]-acetic acid;
[5-Fluoro-2-methyl-3-(quinoxalin-2-ylmethyl)indol-1-yl]-acetic acid;
[5-Fluoro-3-(4-methoxy-benzyl)-2-methyl-indol-1-yl]-acetic acid;
[5-Fluoro-2-methyl-3-(1,3-thiazol-2-ylmethyl)indol-1-yl]acetic acid;
[3-(4-Chloro-benzyl)-5-fluoro-2-methyl-indol-1-yl]-acetic acid;
[5-Fluoro-2-methyl-3-(4-trifluoromethyl-benzyl)-indol-1-yl]-acetic acid;
[5-Fluoro-2-methyl-3-(4-tert-butyl-benzyl)-indol-1-yl]-acetic acid;
{5-Fluoro-2-methyl-3-[(4-phenylphenyl)methyl]indol-1-yl}-acetic acid;
[5-Fluoro-3-(4-methanesulfonyl-benzyl)-2-methyl-indol-1-yl]-acetic acid;
{5-Fluoro-3-[(6-fluoroquinolin-2-yl)methyl]-2-methylindol-1-yl}-acetic acid;
(2-Methyl-3-quinolin-2-ylmethyl-indol-1-yl)-acetic acid;
(5-Chloro-2-methyl-3-quinolin-2-ylmethyl-indol-1-yl)-acetic acid;
(3-{[1-(Benzenesulfonyl)pyrrol-2-yl]methyl}-5-fluoro-2-methylindol-1-yl)-acetic acid;
[5-Fluoro-2-methyl-3-({1-[(4-methylbenzene)sulfonyl]pyrrol-2-yl}methyl)indol-1-yl]-acetic acid;
[3-({1-[(2,4-Difluorobenzene)sulfonyl]pyrrol-2-yl}methyl)-5-fluoro-2-methylindol-1-yl]-acetic acid;
(3-{[2-(Benzenesulfonyl)phenyl]methyl}-5-fluoro-2-methylindol-1-yl)-acetic acid;
[3-({2-[(4-Chlorobenzene)sulfonyl]phenyl}methyl)-5-fluoro-2-methylindol-1-yl]-acetic acid;
[5-Fluoro-3-({2-[(4-fluorobenzene)sulfonyl]phenyl}methyl)-2-methylindol-1-yl]-acetic acid;
(3-{[2-(Benzenesulfonyl)pyridin-3-yl]methyl}-5-fluoro-2-methylindol-1-yl)-acetic acid;
[5-Fluoro-3-({2-[(4-fluorobenzene)sulfonyl]pyridin-3-yl}methyl)-2-methylindol-1-yl]-acetic acid;
[3-({2-[(4-Chlorobenzene)sulfonyl]pyridin-3-yl}methyl)-5-fluoro-2-methylindol-1-yl]-acetic acid;
2-(3-(4-(Benzylsulfonyl)benzyl)-5-fluoro-2-methyl-indol-1-yl)-acetic acid;
2-(3-(4-(4-Chlorobenzylsulfonyl)benzyl)-5-fluoro-2-methyl-indol-1-yl)-acetic acid;
2-(3-(3-(Benzylsulfonyl)benzyl)-5-fluoro-2-methyl-indol-1-yl)-acetic acid;
2-(5-Fluoro-3-(3-(4-fluorobenzylsulfonyl)benzyl)-2-methyl-indol-1-yl)-acetic acid;
2-(3-(2-(Benzylsulfonyl)benzyl)-5-fluoro-2-methyl-indol-1-yl)-acetic acid;
2-(3-(4-(4-Fluorobenzylsulfonyl)benzyl)-5-fluoro-2-methyl-indol-1-yl)-acetic acid;
2-(3-(2-(Cyclohexylsulfonyl)benzyl)-5-fluoro-2-methyl-indol-1-yl)-acetic acid;
2-(5-Fluoro-2-methyl-3-(2-(piperidin-1-ylsulfonyl)benzyl)-indol-1-yl)-acetic acid;
2-(3-(2-(Cyclopentylsulfonyl)benzyl)-5-fluoro-2-methyl-indol-1-yl)-acetic acid;
2-(5-Fluoro-2-methyl-3-(3-(piperidin-1-ylsulfonyl)benzyl)-indol-1-yl)-acetic acid;
2-(5-Fluoro-2-methyl-3-(2-(pyrrolidin-1-ylsulfonyl)benzyl)-indol-1-yl)-acetic acid;
2-(3-(4-(Cyclohexylsulfonyl)benzyl)-5-fluoro-2-methyl-indol-1-yl)-acetic acid;
2-(3-(4-(Cyclopentylsulfonyl)benzyl)-5-fluoro-2-methyl-indol-1-yl)-acetic acid;
2-(3-(2-(Cyclobutylsulfonyl)benzyl)-5-fluoro-2-methyl-indol-1-yl)-acetic acid;
2-(5-Fluoro-2-methyl-3-(3-(pyrrolidin-1-ylsulfonyl)benzyl)-indol-1-yl)-acetic acid;
2-(5-Fluoro-2-methyl-3-(4-(piperidin-1-ylsulfonyl)benzyl)-indol-1-yl)-acetic acid;
[5-Fluoro-2-methyl-3-(2-phenoxybenzyl)-indol-1-yl]-acetic acid;
[5-Fluoro-2-methyl-3-(2-(4-methoxyphenoxy)benzyl)-indol-1-yl]-acetic acid;
[5-Fluoro-2-methyl-3-(2-(4-methylphenoxy)benzyl)-indol-1-yl]-acetic acid;
[5-Fluoro-2-methyl-3-(2-(2,4-dichlorophenoxy)benzyl)-indol-1-yl]-acetic acid;
[5-Fluoro-2-methyl-3-(2-(4-fluorophenoxy)benzyl)-indol-1-yl]-acetic acid;
[5-Fluoro-2-methyl-3-(2-(3,4-difluorophenoxy)benzyl)-indol-1-yl]-acetic acid;
[5-Fluoro-2-methyl-3-(2-(4-cyanophenoxy)benzyl)-indol-1-yl]-acetic acid;
[5-Fluoro-2-methyl-3-(2-(4-chlorophenoxy)benzyl)-indol-1-yl]-acetic acid;
[5-Fluoro-2-methyl-3-(2-(2-cyanophenoxy)benzyl)-indol-1-yl]-acetic acid;
(5-Fluoro-2-methyl-3-{[2-(4-methylphenoxy)pyridin-3-yl]methyl}indol-1-yl)-acetic acid;
{5-Fluoro-3-[(3-methanesulfonylnaphthalen-2-yl)methyl]-2-methylindol-1-yl}-acetic acid;
{5-Fluoro-3-[(1-methanesulfonylnaphthalen-2-yl)methyl]-2-methylindol-1-yl}-acetic acid;
{5-Fluoro-3-[(6-methanesulfonylnaphthalen-2-yl)methyl]-2-methylindol-1-yl}-acetic acid;
[5-Fluoro-2-methyl-3-(quinolin-3-ylmethyl)indol-1-yl]-acetic acid;
[5-Fluoro-2-methyl-3-(quinoxalin-6-ylmethyl)indol-1-yl]-acetic acid;
[5-Fluoro-2-methyl-3-(quinolin-7-ylmethyl)indol-1-yl]-acetic acid;
{5-Fluoro-3-[(6-methanesulfonylquinolin-2-yl)methyl]-2-methylindol-1-yl}-acetic acid;
{5-Fluoro-3-[(4-methanesulfonylquinolin-2-yl)methyl]-2-methylindol-1-yl}-acetic acid;
(5-Fluoro-2-methyl-3-{pyrazolo[1,5-a]pyridin-3-ylmethyl}indol-1-yl)-acetic acid;
(5-Fluoro-3-{imidazo[1,2-a]pyridin-2-ylmethyl}-2-methylindol-1-yl)-acetic acid;
(5-Fluoro-2-methyl-3-{[2-(methylsulfanyl)phenyl]methyl}indol-1-yl)-acetic acid;
(5-Fluoro-2-methyl-3-{[3-(methylsulfanyl)phenyl]methyl}indol-1-yl)-acetic acid;
(5-Fluoro-2-methyl-3-{[4-(ethylsulfanyl)phenyl]methyl}indol-1-yl)-acetic acid;
(3-{[4-(Ethylsulfanyl)phenyl]methyl}-5-fluoro-2-methylindol-1-yl)-acetic acid;
(5-Fluoro-2-methyl-3-{[4-(n-propylsulfanyl)phenyl]methyl}indol-1-yl)-acetic acid;
(5-Fluoro-2-methyl-3-{[4-(i-propylsulfanyl)phenyl]methyl}indol-1-yl)-acetic acid;
(5-Fluoro-2-methyl-3-{[4-(t-butylsulfanyl)phenyl]methyl}indol-1-yl)-acetic acid;
(5-Fluoro-2-methyl-3-{[4-(pentan-3-ylsulfanyl)phenyl]methyl}indol-1-yl)-acetic acid;
[3-({4-[(Cyclopropylmethyl)sulfanyl]phenyl}methyl)-5-fluoro-2-methylindol-1-yl]-acetic acid;
{3-[(4,4-Dimethyl-2,3-dihydro-1-benzothiopyran-6-yl)methyl]-5-fluoro-2-methylindol-1-yl}-acetic acid;
(3-{[2-(Ethanesulfonyl)phenyl]methyl}-5-fluoro-2-methylindol-1-yl)-acetic acid;
(5-Fluoro-2-methyl-3-{[2-(propane-1-sulfonyl)phenyl]methyl}indol-1-yl)-acetic acid;
(5-Fluoro-2-methyl-3-{[2-(propane-2-sulfonyl)phenyl]methyl}indol-1-yl)-acetic acid;
(3-{[2-(Butane-1-sulfonyl)phenyl]methyl}-5-fluoro-2-methylindol-1-yl)-acetic acid;
(3-{[2-(Butane-2-sulfonyl)phenyl]methyl}-5-fluoro-2-methylindol-1-yl)-acetic acid;
(5-Fluoro-2-methyl-3-{[2-(2-methylpropane-2-sulfonyl)phenyl]methyl}indol-1-yl)-acetic acid;
(5-Fluoro-2-methyl-3-{[2-(pentane-1-sulfonyl)phenyl]methyl}indol-1-yl)-acetic acid;
(3-{[2-(Cyclopropylmethane)sulfonylphenyl]methyl}-5-fluoro-2-methylindol-1-yl)-acetic acid;
(5-Fluoro-2-methyl-3-{[2-(propylsulfamoyl)phenyl]methyl}indol-1-yl)-acetic acid;
(3-{[2-(Butylsulfamoyl)phenyl]methyl}-5-fluoro-2-methylindol-1-yl)-acetic acid;
(5-Fluoro-2-methyl-3-{[3-(propylsulfamoyl)phenyl]methyl}indol-1-yl)-acetic acid;
(3-{[3-(Butylsulfamoyl)phenyl]methyl}-5-fluoro-2-methylindol-1-yl)-acetic acid;
(5-Fluoro-2-methyl-3-{[4-(trifluoromethane)sulfonylphenyl]methyl}indol-1-yl)-acetic acid;
(3-{[4-(Ethanesulfonyl)phenyl]methyl}-5-fluoro-2-methylindol-1-yl)-acetic acid;
(5-Fluoro-2-methyl-3-{[4-(propane-1-sulfonyl)phenyl]methyl}indol-1-yl)-acetic acid;
(5-Fluoro-2-methyl-3-{[4-(propane-2-sulfonyl)phenyl]methyl}indol-1-yl)-acetic acid;
(3-{[4-(Butane-1-sulfonyl)phenyl]methyl}-5-fluoro-2-methylindol-1-yl)-acetic acid;
(5-Fluoro-2-methyl-3-{[4-(2-methylpropane-2-sulfonyl)phenyl]methyl}indol-1-yl)-acetic acid;
(5-Fluoro-2-methyl-3-{[4-(pentane-1-sulfonyl)phenyl]methyl}indol-1-yl)-acetic acid;
(5-Fluoro-2-methyl-3-{[4-(pentan-3-ylsulfonyl)phenyl]methyl}indol-1-yl)-acetic acid;
[3-({4-[(Cyclopropylmethyl)sulfonyl]phenyl}methyl)-5-fluoro-2-methylindol-1-yl]-acetic acid;
(5-Fluoro-2-methyl-3-{[4-(propylsulfamoyl)phenyl]methyl}indol-1-yl)-acetic acid;
(3-{[4-(Butylsulfamoyl)phenyl]methyl}-5-fluoro-2-methylindol-1-yl)-acetic acid;
(5-Fluoro-2-methyl-3-{[4-(trifluoromethoxy)phenyl]methyl}indol-1-yl)-acetic acid;
(5-Fluoro-3-{[4-methanesulfonyl-3-(trifluoromethyl)phenyl]methyl}-2-methylindol-1-yl)-acetic acid;
(5-Fluoro-3-{[4-methanesulfonyl-3-(trifluoromethoxy)phenyl]methyl}-2-methylindol-1-yl)-acetic acid;
{5-Fluoro-3-[(5-methanesulfonylthiophen-2-yl)methyl]-2-methylindol-1-yl}-acetic acid;
{3-[(4,4-dimethyl-1,1-dioxo-2,3-dihydro-1λ⁶-benzothiopyran-6-yl)methyl]-5-fluoro-2-methylindol-1-yl}-acetic acid;
[3-({1-[(4-Chlorobenzene)sulfonyl]pyrrol-2-yl}methyl)-5-fluoro-2-methylindol-1-yl]-acetic acid;
[5-Fluoro-3-({1-[(4-fluorobenzene)sulfonyl]pyrrol-2-yl}methyl)-2-methylindol-1-yl]-acetic acid;
[5-Fluoro-3-({1-[(4-methoxybenzene)sulfonyl]pyrrol-2-yl}methyl)-2-methylindol-1-yl]-acetic acid;
{3-[1-(2,4-Dichloro-benzenesulfonyl)pyrrol-2-ylmethyl]-5-fluoro-2-methyl-indol-1-yl}-acetic acid;
[5-Fluoro-3-({1-[(4-methanesulfonylbenzene)sulfonyl]pyrrol-2-yl}methyl)-2-methylindol-1-yl]acetic acid;
{5-Fluoro-2-methyl-3-[(2-phenylphenyl)methyl]indol-1-yl}-acetic acid;
(3-{[1-(Benzenesulfonyl)indol-2-yl]methyl}-5-fluoro-2-methylindol-1-yl)-acetic acid;
(3-{[2-(4-Chlorophenyl)phenyl]methyl}-5-fluoro-2-methylindol-1-yl)-acetic acid;
(5-Fluoro-2-methyl-3-{[2-(4-methylphenyl)phenyl]methyl}indol-1-yl)-acetic acid;
{5-Fluoro-2-methyl-3-[(3-phenoxyphenyl)methyl]indol-1-yl}-acetic acid;
[5-Fluoro-3-({4-[(4-fluorophenyl)carbonyl]-1-methylpyrrol-2-yl}methyl)-2-methylindol-1-yl]-acetic acid;
{5-Fluoro-2-methyl-3-[(6-{[3-(trifluoromethyl)phenyl]methyl}pyridin-3-yl)methyl]indol-1-yl}-acetic acid;
{5-Fluoro-2-methyl-3-[(3-phenoxythiophen-2-yl)methyl]indol-1-yl}-acetic acid;
(3-{[2-(Benzenesulfonyl)-1,3-thiazol-5-yl]methyl}-5-fluoro-2-methylindol-1-yl)-acetic acid;
{3-[(1-Benzylpyrazol-4-yl)methyl]-5-fluoro-2-methylindol-1-yl}-acetic acid;
(3-{[5-(4-Chlorophenoxy)-1-methyl-3-(trifluoromethyl)pyrazol-4-yl]methyl}-5-fluoro-2-methylindol-1-yl)-acetic acid;
[3-({5-[(4-Chlorobenzene)sulfonyl]furan-2-yl}methyl)-5-fluoro-2-methylindol-1-yl]-acetic acid;
[3-({5-[(4-Chlorobenzene)sulfonyl]thiophen-2-yl}methyl)-5-fluoro-2-methylindol-1-yl]-acetic acid;
[3-({3-[(4-Chlorobenzene)sulfonyl]thiophen-2-yl}methyl)-5-fluoro-2-methylindol-1-yl]-acetic acid;
{3-[(2-Benzylphenyl)methyl]-5-fluoro-2-methylindol-1-yl}-acetic acid;
or the C₁-C₆ alkyl, aryl, (CH₂)ₘOC(═O)C₁-C₆alkyl, ((CH₂)ₘO)ₙCH₂CH₂X, (CH₂)ₘN(R⁷)₂, or CH((CH₂)ₘO(C═O)R⁸)₂ esters of any of the above; wherein
m is 1 or 2;
n is 1-4;
X is OR⁷ or N(R⁷)₂;
R⁷ is hydrogen or methyl;
R⁸ is C₁-C₁₈ alkyl.

40. The method of claim 26, wherein the compound of general formula (I) is administered in combination with one or more additional agent which is of use in the treatment of allergic conditions.

41. The method of claim 40, wherein the additional agent is selected from the group consisting of:
Suplatast tosylate and similar compounds;
B₂ adrenoreceptor agonists such as metaproterenol, isoproterenol, isoprenaline, albuterol, salbutamol, formoterol, salmeterol, terbutaline, orciprenaline, bitolterol mesylate, pirbuterol and indacaterol or methylxanthanines such as theophylline and aminophylline, mast cell stabilisers such as sodium cromoglycate or muscarinic receptor antagonists such as ipratropium and tiotropium;
antihistamines, for example histamine H₁ receptor antagonists such as loratadine cetirizine, desloratadine, levocetirizine, fexofenadine, astemizole, azelastine and chlorpheniramine or H₄ receptor antagonists;
α₁ and α₂ adrenoreceptor agonists such as propylhexedrine phenylephrine, phenylpropanolamine, pseudoephedrine, naphazoline hydrochloride, oxymetazoline hydrochloride, tetrahydrozoline hydrochloride, xylometazoline hydrochloride and ethylnorepinephrine hydrochloride;
corticosteroids such as prednisone, prednisolone, flunisolide, triamcinolone acetonide, beclomethasone dipropionate, budesonide, fluticasone propionate, fluticasone furoate, mometasone furoate and ciclesonide; and
allergen immunotherapy such as Grazax.

42. The method of claim 26, wherein the compound of general formula (I) is administered via the oral, nasal, bronchial, or topical route.

43. The method of claim 26, wherein the administration of a compound of general formula (I) provides for the prolonged reduction but not the prevention of the severity of the symptoms.

44. The method of claim 26, wherein the atopic state of the patient is reduced.

45. The method of claim 26, wherein the symptoms of the allergic condition continue to be reduced for at least 7 days after the cessation of treatment with the compound of general formula (I).

46. The method of claim 26, wherein the symptoms of the allergic condition continue to be reduced for at least 35 days after ceasing administration of the compound of general formula (I).

47. The method of claim 44, wherein the reduction of atopic state, as measured either by a reduction in the number of cells reactive to allergens or the level of circulating IgE or the reduction in positive reaction in a skin prick allergy test is at least 30%.

48. The method of claim 44, wherein the reduction of atopic state, as measured either by a reduction in the number of cells reactive to allergens or the level of circulating IgE or the reduction in positive reaction in a skin prick allergy test is at least 60%.
